(19) United States
(12) Patent Application Publication
(10) Pub. No.: US 2003/0069485 A1
(43) Pub. Date: Apr. 10, 2003

(54) Optical image measuring device

(75) Inventors: Ikuo Konishi, Atugi-city (JP); Yoshio Tsunazawa, Hadano-city (JP); Tetsuo Tamai, Columbia, MD (US); Manami Kobayashi, Hadano-city (JP); Yasunobu Ito, Hadano-city (JP)

Correspondence Address: ARMSTRONG,WESTERMAN & HATTORI, LLP, 1725 K STREET, NW, SUITE 1000, WASHINGTON, DC, 20006, US

(73) Assignee: SHIMADZU CORPORATION, Kyoto-shi (JP)

(21) Appl. No.: 09/972,904

(22) Filed: Oct. 10, 2001

(51) Int. Cl. A61B 6/00

(52) U.S. Cl. 600/317

(57) ABSTRACT

An optical image measuring device for obtaining images by irradiating light onto an organism (measurement subject), and detecting the light emitted from the organism, by means of a two-dimensional detector, comprising image measuring means for measuring image data of a plurality of wavelengths, over time; and image calculating means for performing image calculation for determining biological information, using a plurality of image data having respectively different measurement wavelengths and measurement timings. The two-dimensional amount of change in the measurement subject, such as an organism, is obtained as a plurality of image data having different measurement wavelengths and measurement timings, and significant biological information at the measurement timing is acquired by calculating the obtained image data at multiple wavelengths and a plurality of timings.

### BACKGROUND OF THE INVENTION

1. Field of the Invention

The present invention relates to an optical image measuring device for irradiating light inside a living organism, detecting the light emitted by the organism, by means of a two-dimensional detector, and measuring information relating to the organism by means of images obtained from the detector.

2. Description of the Related Art

There are known optical type measuring devices which irradiate light onto a measurement subject, such as an organism, receive the light scattered, reflected and absorbed by the measurement subject, and optically measure the structure of the measurement subject. Various technologies relating to optical measuring devices of this kind have been proposed previously. Prior art examples of optical measuring devices are described below.

When light having wavelengths from visible light to near infrared is irradiated onto an organism, the irradiated light is either absorbed or scattered inside the organism and then emitted from the interior of the organism. There is known a biological monitor which investigates and diagnoses the structure of an organism by receiving this light and measuring the absorption spectrum of the light.

One known type of biological monitor is an oxygen monitor. An oxygen monitor measures relative changes and absolute volumes of oxygenated haemoglobin and de-oxygenated haemoglobin, in a non-invasive manner, by means of the spectral differences between oxygenated haemoglobin (oxy-haemoglobin) which is combined with oxygen, and deoxygenated haemoglobin (deoxy-haemoglobin) which has been separated from oxygen.

In general, in biological measurement, it is not possible to obtain a reference material which has the same optical characteristics as the organism whilst having a known concentration or zero concentration, and consequently, although it is comparatively easy to obtain values for relative change from a value at a specified time, it is difficult to measure absolute values.

Therefore, it has been proposed that an absolute value be obtained by measurement using the outputs of a plurality of detectors positioned at different distances from a light source, based on the assumption that the structure is uniform.

It has also been proposed that images outputs or outputs similar to images be obtained by means of a two-dimensional detector, or a plurality of detectors arranged in a two-dimensional configuration. Furthermore, with regard to the measurement depth at which the optical measurement device takes its measurement, it is known that the measurement depth measured by reflective measurement is dependent on the wavelength and the distance between the light origin and the light reception point.

Optical measurement devices may be required to diagnose the state of blood vessels or blood circulation in a shallow portion of the organism, such as the skin. In order to diagnose whether the blood vessels or blood flow in such a shallow portion of the organism are in a normal state or not, it is necessary to acquire two-dimensional information for the absolute values at the time of measurement, at a shallow measurement depth.

However, in the optical measurement device described above, in a method for determining a two-dimensional image simply by detecting light reflected or transmitted by the measurement subject by means of a two-dimensional detector, the measurement value is a relative value and since no reference value can be obtained, so that the resulting image has no meaning in terms of diagnosing the state of the blood vessels and blood flow.

Furthermore, in order to obtain two-dimensional information using the aforementioned absolute value measurement method, it is necessary to place a plurality of detectors at different distances from the light source and then determine an absolute value for each detector, and hence a problem arises in that the device becomes complex and the calculation time is increased. Moreover, in the aforementioned absolute value measurement, it is assumed that the structure is uniform, and therefore the measurement values are meaningless and accurate diagnosis cannot be performed, if the structure is non-uniform.

### OBJECTS AND SUMMARY OF THE INVENTION

A first object of the present invention is to provide an optical measurement device whereby significant biological information at the time of measurement can be obtained by means of images obtained from the two-dimensional change value of a measurement subject, such as an organism. A second object of the present invention is to provide an optical image measuring device whereby synchronism of measurement can be ensured, by measuring a plurality of images having different optical characteristics, such as wavelength, or the like, without generating time differentials between the measurement timings thereof. Moreover, a third object of the present invention is to provide an optical biological measurement method and measurement device whereby absolute values at the time of measurement can be obtained simply, within a short period of time, by means of two-dimensional image information for the measurement subject as obtained by a single two-dimensional detector.

In order to achieve the first object of the present invention, in a first aspect of the optical image measuring device according to the present invention, the two-dimensional amount of change in a measurement subject, such as an organism, is measured using different measurement wavelengths and measurement timings to obtain a plurality of image data, and significant biological information at the time of measurement is acquired by calculating the obtained image data at multiple wavelengths and a plurality of timings. The biological information is used in diagnosis of a state organization of organism, such as a state of blood vessel and blood flow. The biological information contains information relating, for example, to a blockage wherein a patient's blood vessels become narrowed or restricted.

The optical image measuring device according to the first aspect is an optical image measuring device wherein light is irradiated onto an organism, and the light emitted from the organism is detected by a two-dimensional detector; comprising: image measuring means for measuring image data of a plurality of wavelengths, over time; and image calculating means for performing image calculation for determining biological information, using a plurality of image data having respectively different measurement wavelengths and measurement timings. By means of this optical image measuring device, the two-dimensional amount of change in the measurement subject, such as an organism, is obtained as a plurality of image data having different measurement wavelengths and measurement timings, and significant biological information at the measurement timing is acquired by calculating the obtained image data at multiple wavelengths and a plurality of timings.

The value of the measurement wavelengths and the number of measurement wavelengths of the image data obtained by the image measuring means can be determined according to the biological information to be determined. Furthermore, the calculation details of the image calculation performed by the image calculating means can be determined by a calculation formula set according to the data type and number of data items in the image data determined by the image measuring means.

In order to achieve the second object of the present invention, a second aspect of the optical image measuring device according to the present invention divides the imaging region of a single imaging element into a plurality of areas, and forms images obtained by separating an image to be detected into a plurality of images, on the respective divided imaging areas. By adopting a composition wherein a plurality of images are formed by separating an image to be detected, disparity in the measurement timings of the plurality of images can be reduced and hence synchronism of measurement is ensured. Moreover, since this separation of the image to be detected does not involve any mechanical operation, it is possible to detect a plurality of images at high speed, and hence even in the case of a measurement subject showing rapid change, time change can be detected in a plurality of images without time delay therebetween.

In order to perform the aforementioned measurement, the optical image measuring device according to the second aspect of the present invention comprises an optical system having image separating and forming means and optical characteristics selecting means; and single imaging means for capturing a plurality of images obtained via the optical system, simultaneously or virtually simultaneously. The separating and image forming means separates a single image to be detected into a plurality of images and forms said images on a single imaging means, and the optical characteristics selecting means selectively extracts an optical characteristics with respect to at least one of the plurality of images. Furthermore, the single imaging means is constituted such that the separated and formed plurality of images are detected respectively and independently. By means of this composition, it is possible to measure images at a plurality of wavelengths without producing delay between the measurement timings thereof, and hence measurement synchronism is ensured, and by high-speed acquisition of images at a plurality of wavelengths, it is possible to obtain images of time change for a measurement subject experiencing rapid change, using a plurality of wavelengths.

In order to achieve the third object of the present invention, the optical biological measurement method according to the present invention is a measurement method wherein light is irradiated onto an organism, the light emitted from the organism is detected by a two-dimensional detector, and biological information is measured using the detected images; comprising the steps of: obtaining measurement image data for a plurality of pixels of an organism using a plurality of measurement wavelength and reference image data for at least one pixel using the same measurement wavelengths as the aforementioned measurement wavelengths; and deriving two-dimensional information for absolute quantities relating to biological information by performing calculation for applying different prescribed weightings for each measurement wavelength to the measurement image data and reference image data, and then summing the data.

Moreover, the optical biological measurement device according to the present invention is a measurement device for implementing the optical biological measurement method according to the present invention, being an optical biological measurement device for irradiating light onto an organism, detecting the light emitted from the organism by a two-dimensional detector, and measuring biological information by means of the detected image; comprising: image measuring means for measuring two-dimensional measurement image data for the organism using a plurality of measurement wavelengths; and image calculating means, having reference image data for at least one pixel obtained using the same measurement wavelength as the aforementioned measurement wavelength, for determining two-dimensional information for absolute quantities relating to biological information, by performing calculation for applying different prescribed weightings for each measurement wavelength to the measurement image data and reference image data.

The value of the measurement wavelengths and the number of measurement wavelengths for obtaining reference image data and measurement image data can be determined according to the biological information to be obtained. Furthermore, the calculation details of the image calculation for obtaining the two-dimensional information for the absolute quantities relating to biological information can be determined according to the data type and number of data items in the image data, and the contents of the biological information to be determined.

### BRIEF DESCRIPTION OF THE DRAWINGS

Further objects and characteristics of the present invention described above will become apparent from the following description of the embodiments with reference to the accompanying drawings, wherein:

FIG. 1 is a diagram for illustrating a general view of a first embodiment of an optical image measuring device according to the present invention;

FIG. 2 shows one example of optical detecting means for measuring two wavelengths using a plurality of optical systems;

FIG. 3 shows one example of optical detecting means for measuring two wavelengths by switching a single optical system;

FIG. 4 is a diagram giving a schematic, illustration of the relationship between measurement image data and processing image data;

FIG. 5 is a diagram showing one example of calculation processing performed by image calculating means illustrated in FIG. 1;

FIG. 6 is a diagram wherein the amount of change in oxy-haemoglobin and the amount of change in deoxy-haemoglobin are determined;

FIG. 7 is a diagram wherein the two-dimensional distribution of the amount of change in oxy-haemoglobin and the amount of change in deoxy-haemoglobin are determined;

FIG. 8 is a diagram for illustrating an approximate view of a second embodiment of a optical image measuring device according to the present invention;

FIG. 9 is an approximate diagram for describing a first example of the optical image measuring device according to the second embodiment;

FIG. 10 is an approximate diagram for describing a second example of the optical image measuring device according to the second embodiment;

FIG. 11 is an approximate diagram for describing a third example of the optical image measuring device according to the second embodiment;

FIG. 12 is an approximate diagram for describing a fourth example of the optical image measuring device according to the second embodiment;

FIG. 13 is an approximate diagram for describing a fifth example of the optical image measuring device according to the second embodiment;

FIG. 14 is a diagram for describing image processing using a marker;

FIG. 15 is a diagram illustrating a method for obtaining images separated into a plurality of wavelengths, by switching filters by means of a linear mechanical movement;

FIG. 16 is a diagram illustrating a method for obtaining images separated into a plurality of wavelengths, by switching filters by means of a rotational mechanical movement;

FIG. 17 is a diagram illustrating a method for obtaining images separated into a plurality of wavelengths, by switching filters by means of a rotational mechanical movement;

FIG. 18 is a diagram illustrating a method for obtaining images separated into a plurality of wavelengths, by switching the light path;

FIG. 19 is a diagram illustrating a method for obtaining images separated into a plurality of wavelengths, by switching the light source;

FIG. 20 is a diagram for describing an overview of an optical biological measurement method according to the present invention;

FIG. 21 is a diagram for describing an overview of a method for making optical biological measurements using two wavelengths;

FIG. 22 is an approximate diagram of the wavelength spectra of oxy-haemoglobin and deoxy-haemoglobin;

FIG. 23 is a diagram for describing an example where reference image data is determined in optical biological measurement;

FIG. 24 is a diagram for describing a first example of a combination of reference image data and measurement image data;

FIG. 25 is a diagram for describing a second example of a combination of reference image data and measurement image data;

FIG. 26 is an example wherein the absolute values of oxy-haemoglobin and deoxy-haemoglobin have been converted into images by an optical biological measurement;

FIG. 27 is a diagram showing an example of Monte Carlo simulation carried out for a skin model, describing an absorbance of human skin calculated by Monte Carlo simulation: and FIG. 28 is a graph explaining the difference of linear and non-linear determination.

### DESCRIPTION OF THE PREFERRED EMBODIMENTS

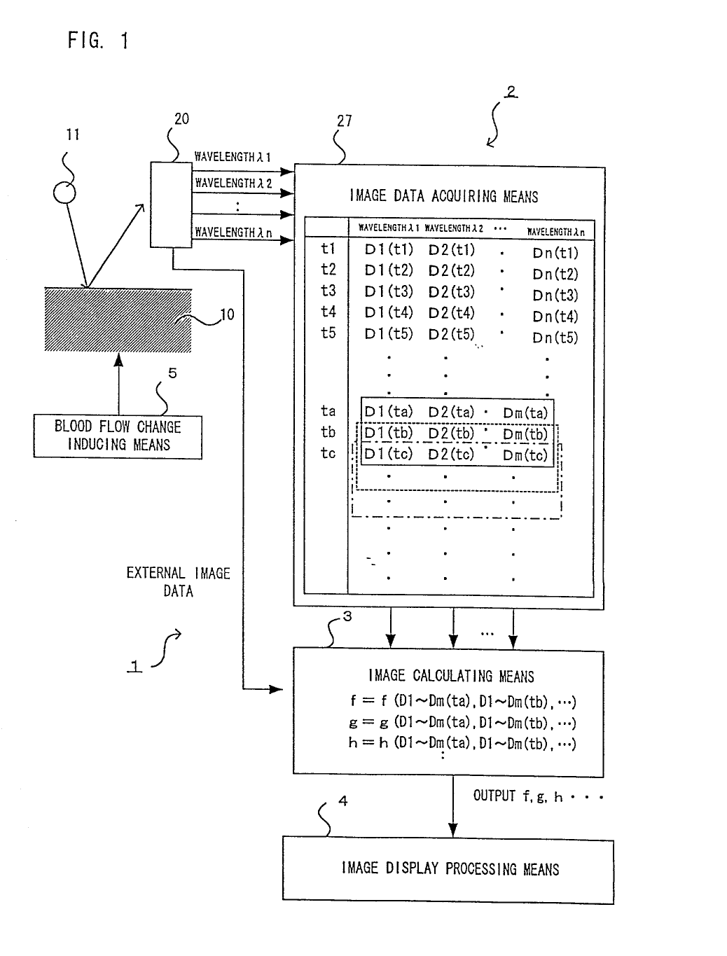

Firstly, a first embodiment of the optical image measuring device according to the present invention is described with reference to FIG. 1 to FIG. 7.

As shown in FIG. 1, the optical image measuring device 1 according to the present embodiment comprises image measuring means 2, image calculating means 3 and image display processing means 4 for displaying the acquired images. Moreover, it is also possible to provide blood flow change inducing means 5 for inducing change in the state of the blood flow of the organism, thereby altering the light emitted from the organism.

The image measuring means 2 detects the light emitted by the measurement subject 10 due to irradiation by a light source 11, by means of a two-dimensional detector, or the like, and it is provided with optical detecting means 20 for outputting image data of a plurality of wavelengths (wavelength λl to wavelength λn) and image data acquiring means 27 for acquiring this image data in a time series fashion.

Image data acquiring means 27 acquires image data D1(t1) D2(t1), . . . , Dn(t1) for wavelengths λ1, λ2, . . . , λn, as image data in a time series of t1, t2, . . . , tn, and the image calculating means 3 performs calculation processing on the basis of this time series image data.

Here, the image data D1(t1), D2(t2), . . . are not single values, but rather, they represent an array of pixels. For example, in the case of an image comprising 100×100=10,000 pixels, D1(t1) will essentially comprise 10,000 individual values for all of picture elements represented by the 100 column×100 row array. However, in order to simplify the notation, here, D1(t1) is taken as a single representative pixel in the array. Therefore, whenever D1(t1) appears in the calculation, it should be noted this indicates that there are pixel as many as the total number of the pixels in the array.

The calculations f, g, h are examples of image calculations performed by the image calculating means 3, and these typically involve calculations to determine the time rate of change of the image, and from this, to derive biological information, by using a plurality of image data having different measurement timings relating to a plurality of wavelengths, from the time series image data provided in the image data acquiring means 27. For example, calculations f, g, h are calculations whereby the image data at timings ta, tb, tc are taken as variables with respect to the wavelengths λ1, λ2, . . . , λm.

The image calculations defined by f, g, h are performed at successively separated timings, in groups of three times (ta, tb, tc), for example, firstly, at (t1, t2, t3), then at (t2, t3, t4), and then at (t3, t4, t5).

If, for instance, the amount of change of oxy-haemoglobin (oxygenated haemoglobin) and the amount of change of deoxy-haemoglobin (deoxygenated haemoglobin), in an organism, are to be acquired as basic quantities relating to biological information, then the image calculating means 2 measures two-wavelength image data over time. As shown in equations (1), (2), (3) below, the image calculating means 3 derives the oxy-haemoglobin change and the deoxy-haemoglobin change by applying a prescribed weighting to each of the four image data values obtained from the two-wavelength image data at two timings, and then carrying out calculation processing to add or subtract these values.

[ΔOxyHb], [ΔdeOxyHb] at first timing

[ΔOxyHb]=k1×D1(t2)+k2×D2(t2)−k1×D1(t1)−k2×D2(t1)

[ΔdeOxyHb]=k3×D1(t2)+k4×D2(t2)−k3×D1(t1)−k4×D2(t1) (1)

[ΔOxyHb], [ΔdeOxyHb] at second timing

[ΔOxyHb]=k1×D1(t3)+k2×D2(t3)−k1×D1(t2)−k2×D2(t2)

[ΔdeOxyHb]=k3×D1(t3)+k4×D2(t3)−k3×D1(t2)−k4×D2(t2) (2)

[ΔOxyHb], [AdeOxyHb] at third timing

[ΔOxyHb] k1×D1(t4)+k2×D2(t4)−k1×D1(t3)−k2×D2(t3)

[ΔdeOxyHb]=k3×D1(t4)+k4×D2(t4)−k3×D1(t3)−k4×D2(t3) (3)

Here, D1(t) is a value representing one pixel of the image data at timing t and wavelength λ1, and D2(t) is a value representing one pixel of the image data at timing t and wavelength λ2.

In the equations (1), (2), (3) above, [ΔOxyHb] is the pixel value of the amount of change of the oxy-haemoglobin, and [ΔdeOxyHb] is the pixel value of the amount of change of the deoxy-haemoglobin. As shown in equation (1), the value of [ΔOxyHb] at the first timing is obtained by applying the weighting coefficients (−k1, −k2, k1, k2) to the four pixel values D1(t1), D2(t1), D1(t2), D2(T2) obtained from the total of four measurement images taken for the two wavelengths at two timings, t1, t2, and then adding the results, Moreover, the value of [ΔdeOxyHb] at the first timing is obtained by applying the weighting coefficients (−k3, −k4, k3, k4) to the same original pixel values D1(t1), D2(t1), D1(t2), D2(T2) and then adding the results.

The values at the second and third timings can be derived, as shown in equations (2) and (3), respectively, by successively shifting the timings (t1, t2), to (t2, t3) and then (t3, t4). The original pixel values D1(t1), D2(t1), and so on, can be obtained from output values of a two-dimensional detector, the valve after dark current subtraction or from a detector output converted to logarithmic values.

Moreover, in order to apply the aforementioned calculation to each pixel, the obtained image is an image wherein each pixel represents the amount of change of oxy-haemoglobin or deoxy-haemoglobin.

One mode of processing implemented by the image calculating means is an image of the time rate of change of biological information at a specified timing, using the image data as described above. In this case, the image of the time rate of change can be derived by calculation for determining differential values for the image data, or by calculation for determining the differences in image data taken as adjacent timings.

In a further mode of processing implemented by the image calculating means, the different timings at which the time rate of change of the image data reaches a maximum, for different wavelengths, are displayed in the form of an image. In other words, the value of the phase delay is converted to an image. For example, it has been found that in a location where there is a vascular blockage, or the like, in the blood vessel distribution, the change in the oxy-haemoglobin is delayed compared to other normal locations. Therefore, by detecting this phase change, it is possible to diagnose a biological state, such as a vascular blockage.

In a further mode of processing implemented by the image calculating means, a maximum value for the amplitude of each image data value constituting the image data, or a maximum value for the differential amplitude of each image data value, is found, the amplitude value or differential amplitude for each image is then normalized with respect to this maximum value, and an image is created using the normalized values for each pixel. Thereby, it is possible to detect even small amplitude changes in the pixels, without fail.

The blood flow change inducing means is means for inducing change in the state of the blood flow in the organism, it being possible to use, for example, a cuff wound about the arm in order to halt blood flow, a heater in order to increase the skin temperature, ice or cooling means in order to reduce the skin temperature, a device for applying a light electrical stimulus to the nerves, and the like, whereby it is possible to restrict or free the blood flow by means of a cuff, provide stimuli by applying temperature, pressure, electricity, light radiation, medical coverings, or the like, to the skin, inject medicines, and the like.

The inventors conducted experimentation wherein a sudden change in the flow of blood in a blood vessel was effected by restricting the upper side of an observation region by means of a cuff, or the like, in order to halt the blood flow intentionally, and then releasing the cuff, and the resulting change in the amount of oxygenated haemoglobin in the skin was displayed as images by means of the optical image measuring device according to the present embodiment. As a result, it was possible to observe differences in the patterns of change in the amount of oxygenated haemoglobin, in terms of the presence or absence of change, the magnitude of the change, and the response speed of the change, in different regions of the skin. These patterns of change relate to the distribution of blood vessels supplying oxygen to the skin, and if a blocked region is created in the blood vessels, then flow of blood will reduce, and hence a significant reduction in the amplitude of change of the oxygenated haemoglobin in the blocked region can be observed in the image.

The blood flow change inducing means intentionally changes the flow of blood in the body tissue, and this change in the state of the blood flow is detected by the optical image measuring device in the form of image data and an image relating to biological information is calculated from this image data. It is possible to distinguish the correct or incorrect functioning of blood vessels in body tissue from the patterns of change thus derived.

Moreover, in one modification example of the optical image measuring device according to the present embodiment, an external image is created on the basis of one image data of the multiple-wavelength image data measured by means of an optical image measuring device according to the first embodiment, and this external image is displayed in a manner such that it is superimposed with images derived from the image data having a plurality of wavelength and a plurality of timings. By means of this display, a merit can be obtained in that the location of the biological information is readily recognized. In this case, since only one element of the original multiple-wavelength image is used, a major benefit is obtained in that an external image and functional images can be combined, without requiring additional hardware devices.

By means of the optical image measuring device according to the present embodiment, it is possible to display images of the activity distribution of oxygen supply to different regions of body tissue, and hence malfunctions, such as blockages of blood vessels, or the like, can be identified from the resulting patterns.

By means of the optical image measuring device according to the present embodiment, since neither X rays nor image forming agents are required, it is possible to reduce the effects on the body, and hence it is possible to achieve a measuring device suitable for repeated measurement, for example, in judging clinical effects.

By means of the optical image measuring device according to the present embodiment, it is possible to obtain two-dimensional image data by adopting a two-dimensional detector, and hence a plurality of light transmitters and receivers are not required and the device can be compactified, in addition to which the price of the device can be reduced and furthermore, since no medicines are used in measurement, the cost of investigation can be reduced.

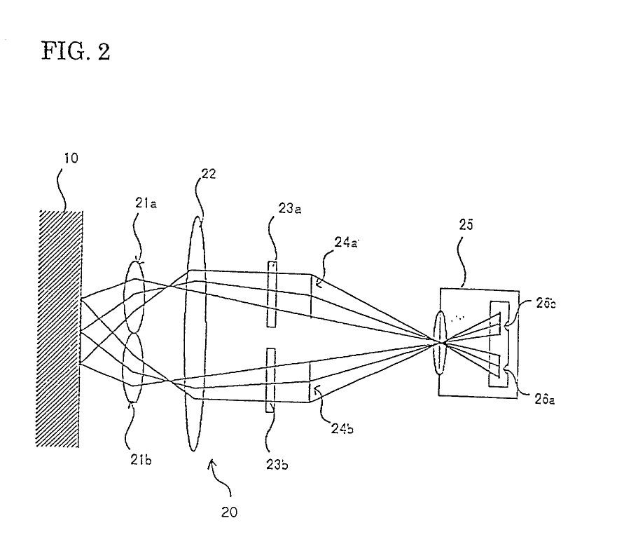

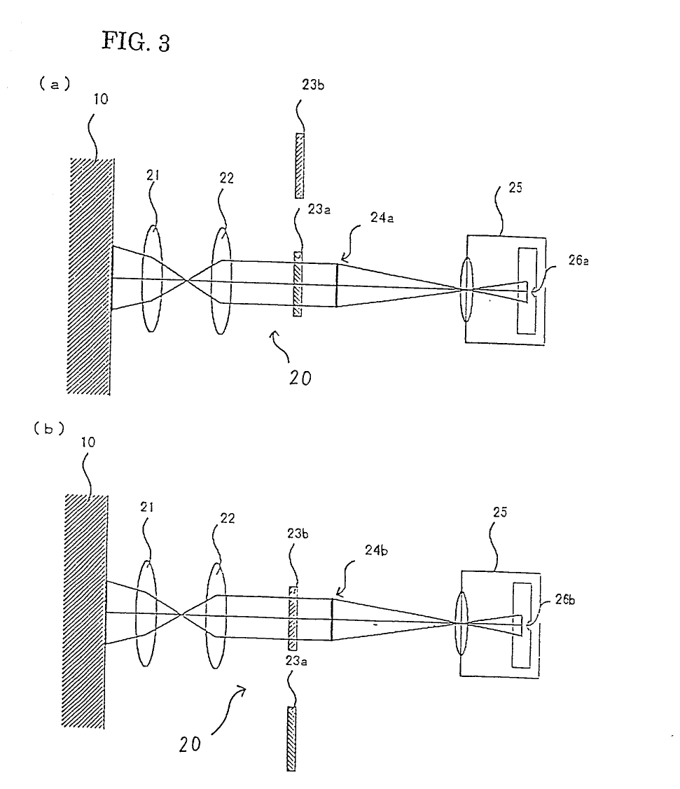

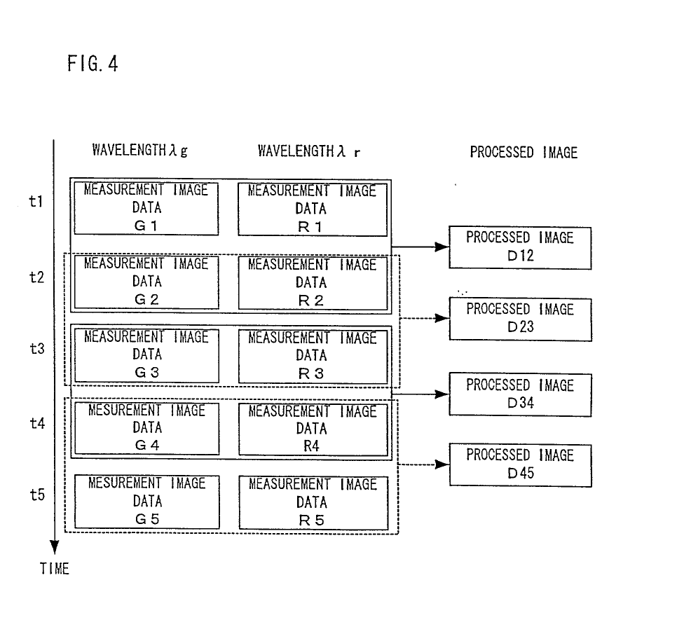

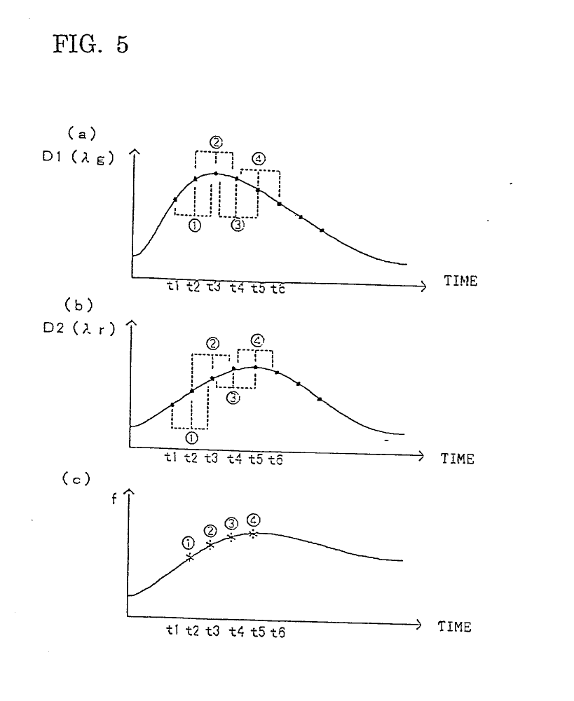

Below, the composition of the optical detecting means 20 used in the optical image measuring device according to the present embodiment is described with reference to FIG. 2 and FIG. 3, and an example of calculation performed by the image calculating means 3 is described with reference to FIG. 4 and FIG. 5.

FIG. 2 and FIG. 3 are examples of optical detecting means in a case where two wavelengths are measured. FIG. 2 is an example where a plurality of optical systems are used, and FIG. 3 is an example where a single optical system is switched.

In the first example shown in FIG. 2, the optical detecting means 20 comprises a lens system 21a, 21b for branching light emitted from the measurement subject 10 into two light paths, a lens system 22 for directing the two branched light paths to a CCD camera 25, and a first wavelength filter 23a and a second wavelength filter 23b for respectively separating a first wavelength components and a second wavelength component from the two branched light paths.

Respective intermediate images 24a, 24b of the respective wavelength components extracted by the first wavelength filter 23a and the second wavelength filter 23b are received by a first wavelength photoreceptor section 26a and a second wavelength light photoreceptor section 26b on a CCD camera 25. These photoreceptor sections 26a, 26b can be constituted respectively by two-dimensional detectors, image data being obtained according to the light intensity detected at each pixel.

In the second example shown in FIG. 3, the optical detecting means 20 comprises the first lens systems 21, 22 guiding the light emitted from the measurement subject 10, the first wavelength filter 23a and second wavelength filter 23b which respectively separate out a first wavelength component and a second wavelength component, and the CCD camera 25. The first wavelength filter 23a and the second wavelength filter 23b is constituted in such a manner that they can be inserted respectively and independently into the light path.

FIG. 3(a) illustrates a case where the first wavelength filter 23a is introduced into the light path, and FIG. 3(b) illustrates a case there the second wavelength filter 23b is introduced into the light path. The intermediate image 24a (FIG. 3(a)), or intermediate image 24b (FIG. 3(b)) of the respective wavelength components extracted by the first wavelength filter 23a or the second wavelength filter 23b are received by the photoreceptor sections 26a, 26b on the CCD camera 25. The photoreceptor sections 26a, 26b are constituted by two-dimensional detectors, and image data is acquired according to the light intensity detected for each pixel. The first wavelength filter 23a and second wavelength filter 23b can be introduced into the light path by moving them orthogonally with respect thereto.

FIG. 4 and FIG. 5 are diagrams for describing image calculation in a case where two-wavelength measurement is used.

Taking the measurement timing as t1, t2, t3, t4, t5, the signal values at each timing for each pixel of the image data constituted by the wavelength component having wavelength λg are taken respectively as G1, G2, G3, G4, G5, . . . , and the signal values at each timing for each pixel for the image data constituted by the wavelength component having wavelength λr are taken respectively as R1, R2, R3, R4, R5, . . . .

Here, it should be noted that the signal values for each pixel are indicated for representative pixels of the CCD, and in a 512×600 CCD, for example, each one of approximately 300,000 pixels will have its own respective signal values corresponding to the received image.

The respective magnitudes of the signal value Gi of the pixel at wavelength λg and the signal value Ri of the pixel at wavelength λr are integers in the range of 0≦(Gi, Ri)≦4095 (=2¹²), in the case of a 12-bit signal; when the values of Gi, Ri are small, then this indicates weak light intensity, and when the values of Gi, Ri are large, this indicates strong light intensity.

To obtain the processed image, calculation is performed using a combination of four image data having different measurement wavelengths and different measurement timings, namely, the image data Gi of the first wavelength λg and the image data Ri of the second wavelength λr at timing i, and the image data Gj of the first wavelength λg and the image data Rj of the second wavelength λr at timing j, this calculation yielding one set of image data.

It should be noted that timing i and timing j are different timings, wherein, for example, timing i is taken as a certain measurement timing, and timing j is taken as the next measurement timing.

FIG. 4 gives a schematic illustration of the relationship between the measurement image data and the processed image data. In FIG. 4, the processing image data D12 is derived by applying calculation processing such as formula (1) described above, to the signal values G1, R1 at timing t1, and the signal values G2, R2 at timing t2 (combination of four measurement image data elements enclosed by solid lines in the diagram). Next, the processing image data D23 is derived by applying calculation processing such as formula (1) described above, to the signal values G2, R2 at timing t2, and the signal values G3, R3 at timing t3 (combination of four measurement image data elements enclosed by dotted lines in the diagram). Thereafter, processed image data D34, D45, are determined similarly by performing calculations using combinations of measurement image data having different wavelengths and different timings.

By using the first wavelength λg of 575 nm (red light) and the second wavelength λr of 600 nm (red light), it is possible to determined the amount of change in the oxy-haemoglobin and deoxy-haemoglobin from the image data Gi and Ri.

The amount of change in the oxy-haemoglobin [AOxyHb] and the amount of change in the deoxy-haemoglobin [AdeOxyHb] are respectively expressed by the following equations, using the image data Gi, Gj, Ri, Rj.

[ΔOxyHb]=k1(log Gj−log Gi)+k2(log Rj−log Ri) (4)

[ΔdeOxyHb]=k3(log Gj−log Gi)+k4(log Rj−log Ri) (5)

k1 to k4 are coefficients derived from the spectra of oxy-haemoglobin and deoxy-haemoglobin, and are set to appropriate values, such as k1=−79, k2=212, k3=20, k4=−322, for example.

FIG. 5 is a diagram giving a conceptual illustration of the processing calculation. FIG. 5 shows a conceptual illustration of calculation for obtaining image data relating to a body organism from the rate of change of the measurement image data, in a similar way as in the case of the calculation of the change in oxy-haemoglobin and deoxy-haemoglobin described above, using a combination of four measurement image data.

FIG. 5(a) shows values for each pixel D1 (λg) of the image at the first wavelength, at respective timings (t1, t2, t3, t4, t5, t6, . . . ), and FIG. 5(b) shows values for each pixel D2 (λr) of the image at the second wavelength, at respective timings (t1, t2, t3, t4, t5, t6, . . . ). FIG. 5(c) shows values for each pixel of the image, at respective timings (t2, t3, t4, t5, t6, . . . ), as obtained by calculation.

In this case, the value at time point \mathring{1} corresponding to the timing t2 in FIG. 5(c) is obtained by calculation of a total of 6 data elements, consisting of the three data marked by the broken line in \mathring{1} of FIG. 5(a) (data at timings t1, t2, t3), and the three data marked by the broken line in \mathring{1} in FIG. 5(b) (data at timings t1, t2, t3).

Similarly, the value at time point \mathring{2} corresponding to timing t3 in FIG. 5(c) is obtained by calculation of a total of six data elements for timings t2, t3, t4, as marked by \mathring{2} in FIG. 5(a) and also in FIG. 5(b). Thereafter, it is possible to obtain an image series corresponding to the rate of change, by proceeding to calculate values at one-step intervals, as shown in \mathring{3}, \mathring{4}, . . . .

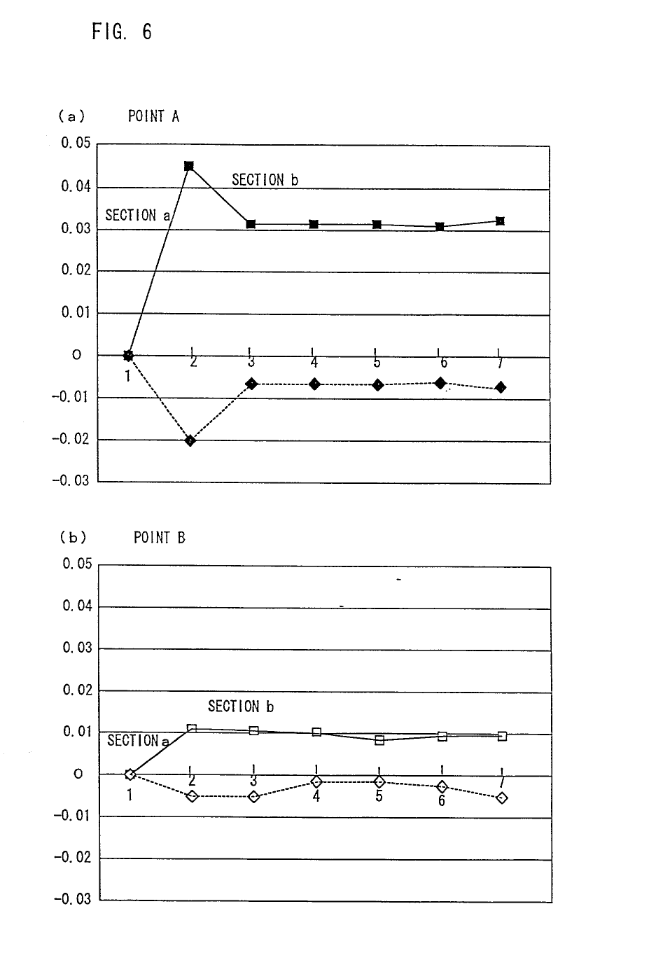

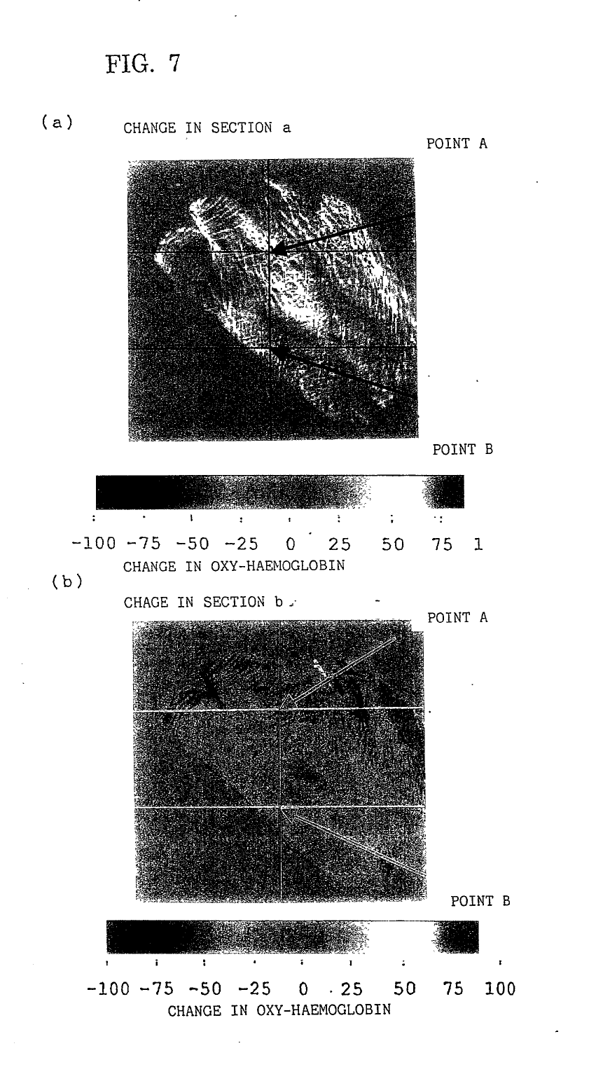

Next, FIG. 6 and FIG. 7 show examples wherein the amount of change of oxy-haemoglobin and the amount of change of deoxy-haemoglobin are obtained using calculation formulae (4) and (5) described above, in the optical image measuring device according to the present embodiment.

In FIG. 6, the amount of change of oxy-haemoglobin is indicated by the broken line and the amount of change of deoxy-haemoglobin is indicated by the solid line. Moreover, FIG. 6 shows the amount of change of oxy-haemoglobin and deoxy-haemoglobin at two points (point A and point B in FIG. 7) on the same measurement subject, with respect to an initial value of zero. FIG. 6(a) shows the amount of change at point A and FIG. 6(b) shows the amount of change at point B.

In FIG. 6, data at time point 1 represents a value before the arm portion of the body under measurement is restricted by a cuff, and data at time point 2 represents a value two minutes after the cuff starts to be tightened. After time point 2, the cuff is released.

From the amount of change in oxy-haemoglobin (broken line) and the amount of change in deoxy-haemoglobin (solid line) shown in FIG. 6, it can be observed that the most highly deoxygenated state is reached at time point 2, that the body rapidly returns towards an oxygenated state when the cuff is released, and that a gentle change is experienced between time point 3 and time point 7. In section a of FIG. 6 (the section between time point 1 and time point 2), the deoxy-haemoglobin increases significantly, and the oxy-haemoglobin decreases significantly. Furthermore, in section b (section between time point 2 and time point 3), it returns rapidly to an oxygenated state.

Moreover, by comparing the change at point A illustrated in FIG. 6(a) and the change at point B illustrated in FIG. 6(b), it is possible to observe differences between regions of the measurement subject.

FIG. 7 is an image showing the differences in oxy-haemoglobin change at respective locations, during section a and section b in FIG. 6. FIG. 7(a) is a two-dimensional distribution image of oxy-haemoglobin in section a, and FIG. 7(b) is a two-dimensional distribution image of oxy-haemoglobin in section b. In FIG. 7(a), there is an overall decline in the oxy-haemoglobin, and in FIG. 7(b), conversely, there is an increase thereof, but in addition to this, there are marked differences between locations. At point A which is a representative comparison point, the range of variation in the oxy-haemoglobin is large, whereas conversely, at point B the range of variation in the oxy-haemoglobin is small. FIG. 6(b) shows that in section b, the oxy-haemoglobin increases in the region of point A, whilst there is little change in the oxy-haemoglobin in the region of point B. Therefore, it can be inferred that when the measurement subject has received a stimulus, the blood flow changes significantly in the region of point A, whereas there is little change in the blood flow in the region of point B, and it is possible to achieve an image display of the activity distribution of the oxygen supply to different regions of the body tissue, and hence abnormalities, such as blood vessel blockages, or the like, can be identified from the resulting patterns.

In general, when the blood flow is slow, the amplitude of change in the measurement values is small, and hence this change is not readily visible when displayed in the form of images. Therefore, it is possible to obtain good imaging, even when the absolute value of the amplitude is small, by storing a series of image data indicating time change, and then normalizing values by, for example, dividing the values obtained at each timing by the maximum value for the pixels contained in all of the image data.

Furthermore, in order to eliminate noise, in the case of pixels having a small amount of change, a uniform threshold value is set for the maximum amplitude and the values is be normalized by means of this uniform threshold value, instead of the maximum value, if the maximum value of the signal for the present pixel does not exceed this threshold value. Thereby, degradation of the image due to noise can be prevented, and regions which change due to a phase delay can be extracted readily.

Moreover, by means of the optical image measuring device according to the present embodiment, it is possible to derive an external image of the measurement subject by using the output of one wavelength from among the multiple-wavelength outputs of the two-dimensional detectors, and hence the region experiencing change in blood flow can be recognized readily by superimposing this external image with images of blood flow change, or the like. For example, it is possible to superimpose an external image based on a contrasting black and white image with images of variation in the state of oxygenation based on artificial colours.

In general, a plurality of images gathered by different measurements are displayed in a superimposed fashion, but in the present embodiment, it is possible to obtain images for different purposes by means of the same device by selecting the wavelength and to display those images in superimposed fashion.

It is also possible use an external image obtained by removing the filter, or an external image obtained while the filter is installed.

As described above, in the optical image measuring device according to the present embodiment, the state of body tissue can be diagnosed by gathering significant biological information during measurement, using image information obtained from two-dimensional amounts of change, without measuring absolute values.

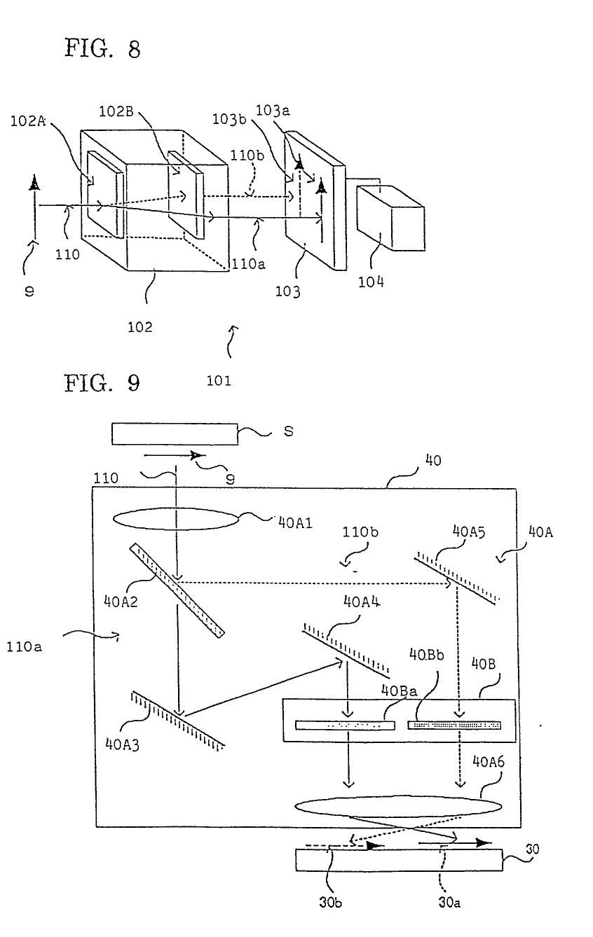

Next, a second embodiment of an optical image measuring device according to the present invention is described with reference to FIG. 8 to FIG. 19.

This optical image measuring device which observes a detected image using a plurality of wavelengths and then performs calculation processing on the observed image has a composition wherein images of a plurality of wavelengths are obtained by mechanically exchanging an optical element, such as a wavelength filter, or the like, and incorporating optical elements which physically switch the light path.

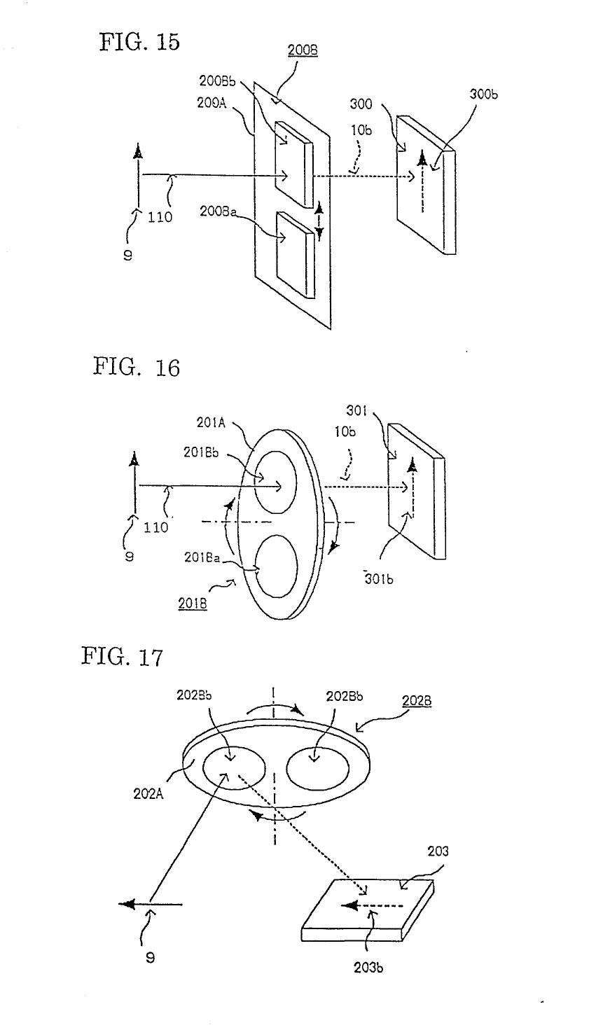

FIG. 15 to FIG. 19 are approximate diagrams for describing compositional examples for optically separating a single image into a plurality of wavelengths, in an optical image measuring device.

Separation of a single image into a plurality of wavelengths can be realized by changing filters having different optical properties, or by changing light sources. And changing of filters is realized by moving the filters mechanically, or by changing light paths.

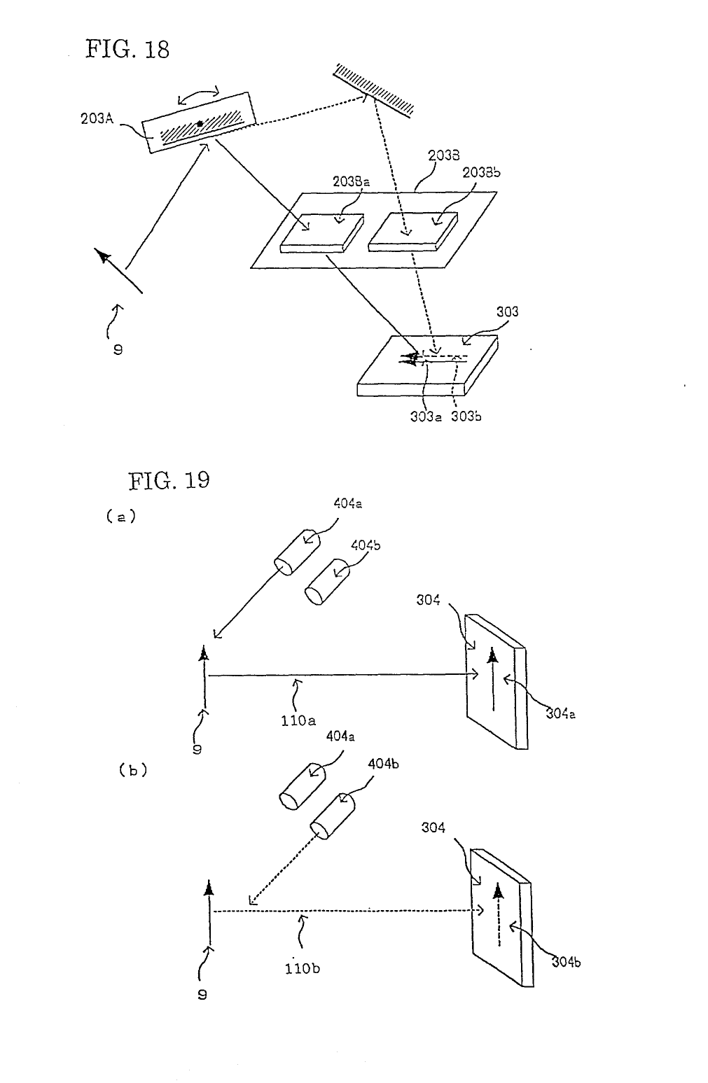

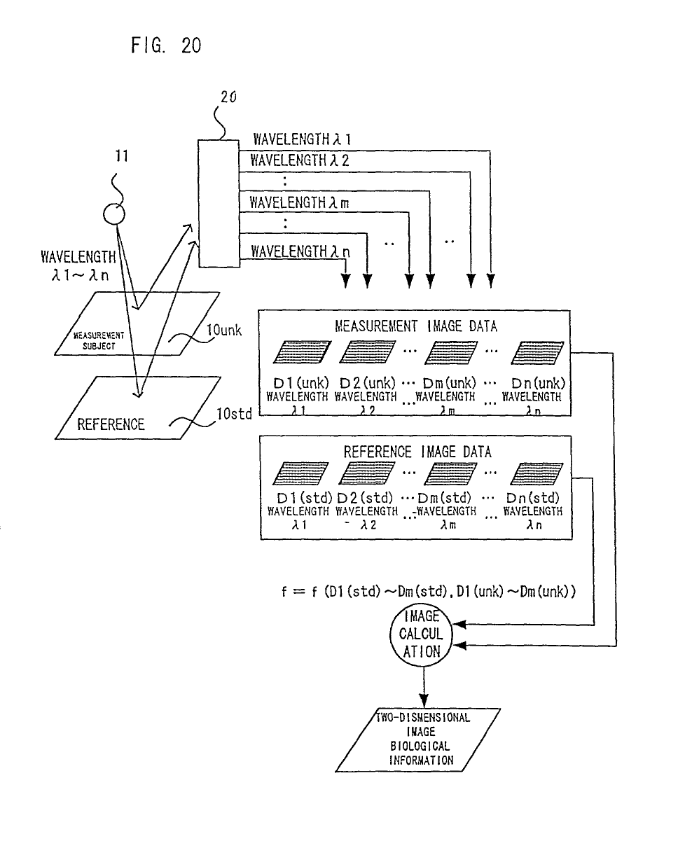

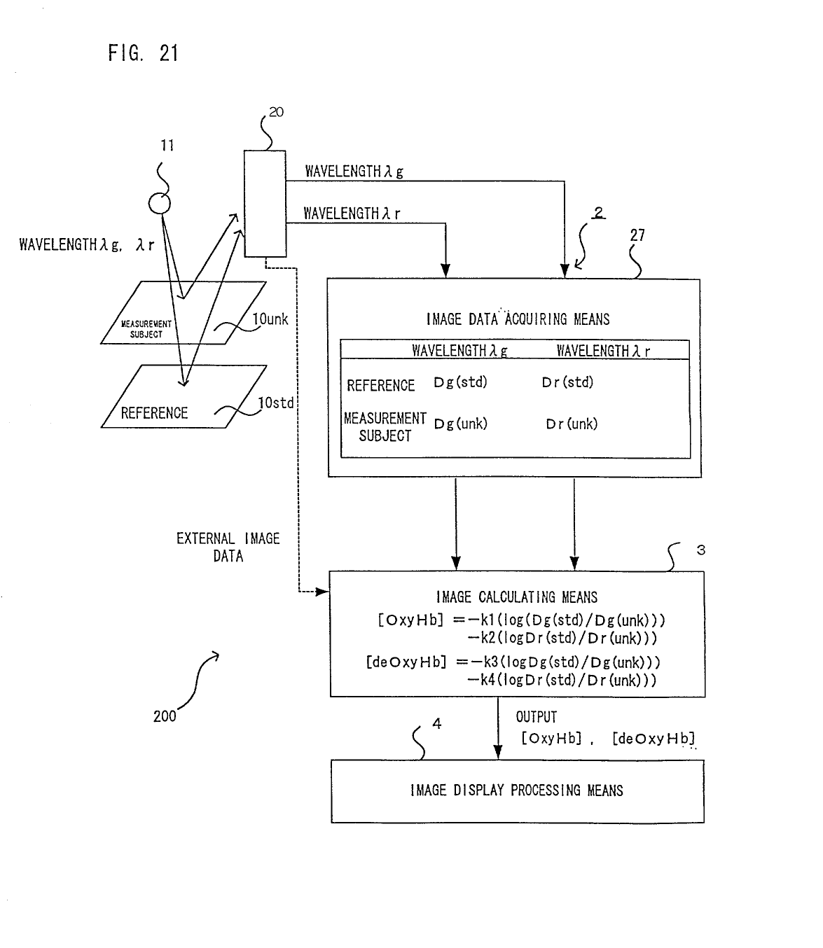

FIG. 15 to FIG. 17 illustrate methods for obtaining an image separated into a plurality of wavelengths, by switching filters by means of mechanical movement; FIG. 18 illustrates a method for obtaining an image separated into a plurality of wavelengths, by switching light path; and FIG. 19 illustrates a method for obtaining an image separated into a plurality of wavelengths, by switching light source.

In the filter switching method shown in FIG. 15, a filter 200B comprising filters 200Bb, 200Ba having optical characteristics corresponding to the measurement wavelengths are moved mechanically and linearly by means of a movement mechanism 200A, with respect to the light path 110, whereby the image formed on the imaging means 300 is exchanged. FIG. 15 shows a state where an image 300b is obtained by means of filter 200B. Moreover, as a drive source for the movement mechanism which performs the linear movement, it is possible to use a hydraulic drive mechanism, a solenoid, or the like. Numeral 9 denotes the image being detected.

In the filter switching method illustrated in FIG. 16, a filter 201B comprising filter 201Bb and 201Ba having optical characteristics corresponding to the measurement wavelengths in an circumference, is rotated mechanically by a rotating mechanism 201A with respect to the light path 110, and with this switching of the filter 201B, the image formed on the imaging means 301 is changed. FIG. 16 illustrates a state where image 301b is obtained by means of filter 201B.

The reflective type filters 202Ba and 202Bb can be switched by rotational movement using a rotating mechanism 202A, as illustrated in FIG. 17. As a drive source for the movement mechanism performing this rotational movement, it is possible to use, for example, continuous rotation or intermittent rotation provided by a DC or AC motor, or intermittent rotation provided by a stepping motor, solenoid, hydraulic motor, or the like.

In the filter switching method shown in FIG. 18, the filter 203B comprising the filters 203Ba and 203Bb having optical properties corresponding to the measurement wavelengths is fixed, and the light path is changed by changing the angel of a switching mirror 203A, thereby directing the light through filter 203Ba or 203Bb and hence switching the image to be formed on the imaging means 303. In FIG. 18, the solid lines indicate an image 303a formed by means of filter 203Ba, and the broken lines indicate an image 303b formed by means of filter 203Bb. As a drive source for the angle changing mechanism, it is possible to use a hydraulic or solenoid-based mechanism, or a motor or cam-based oscillating mechanism, for example.

The light source switching method shown in FIG. 19 involves switching between light sources 404a and 404b having different wavelengths, whereby the image 9 to be detected is formed on the imaging means 304 by irradiation of light of a particular wavelength, through of a pulse light source switching drive, or a combination of a continuously driven light source and a shutter, or the like. FIG. 19(a) shows a case where an image 304a is formed by means of light source 404a, and FIG. 19(b) shows a case where an image 304b is formed by means of light source 404b.

Moreover, the filter switching may also be achieved by means of a liquid crystal switch.

As described above, according to the optical image measuring device illustrated in FIG. 15 to FIG. 19, in a composition where one image is optically separated into a plurality of wavelengths to obtain separated images, the wavelength switching is performed with time, and hence the measurement time point of the respective images of various wavelengths thus obtained will be different, rather than being the same. Consequently, a problem arises in that synchronism of measurement cannot be guaranteed. If the change in the measurement subject is rapid, then time changes will be added between the images measured using a plurality of wavelengths, and it may be difficult to make comparisons between wavelengths. A further problem arise in that high-speed imaging is difficult to achieve, because time is required to change the filters, or the like.

A conceivable method for resolving the aforementioned problems would be to adopt a composition using a plurality of imaging means, but if a plurality of imaging means were to be used, measurement errors may emerge due to performance variations between the imaging elements of the respective imaging means, and the cost of the optical image measuring device would also rise.

Changing of a plurality of light sources entails other problems. In a composition using a plurality of light sources, the positional relationships between the light sources and the measurement subject are not equal, and hence the image properties, such as reflectivity, or the like, will change with variation in the direction of illumination, thereby making it difficult to make comparisons between images. Also, in a composition using a liquid crystal switch to change filters, deflector elements are required before and after the liquid crystal switch in the light axis, and therefore variation in image properties due to the deflector elements must be taken into account.

These various problems, such as variation between imaging elements, changes in illumination direction due to switching light source, changes in image properties due to deflector elements, and the like cause variation in the imaging direction on the imaging surface. This difference in imaging direction corresponds to difference in the observation direction at the image being detected.

Therefore, it is an object of a second embodiment of the optical image measuring device according to the present invention to resolve the aforementioned problems by ensuring synchronism of measurement by measuring a plurality of images having different optical characteristics, such as wavelength, or the like, in such a manner that no time delay occurs between the respective measurement timings thereof when using the optical image measuring device. Furthermore, it is also an object thereof to obtain a plurality of images at high-speed, and to obtain a plurality of images which only differ in terms of a desired optical characteristic, with respect to time change of a measurement subject which experiences rapid change.

An overview of a second embodiment of an optical image measuring device according to the present invention is now described with reference to FIG. 8.

In FIG. 8, the optical image measuring device 101 comprises an optical system 102 and imaging means 103. The optical system 102 comprises separating and image forming means 102A and optical characteristics selecting means 102B. The imaging means 103 comprises a single imaging element. The image projected by the imaging means 103 is subjected to image data processing by the image processing means 104.

The image 9 to be detected is guided via the light 110 to the optical system 102. The optical system 102 branches the image 9 to be detected into a plurality of light paths (first light path 110a, second light path 110b), which generate images in the form of a first image 103a and a second image 103b on the imaging surface of the imaging means 103.

The separating and image forming means 102A is an optical system for separating a single image 9 to be detected into a plurality of images, which are formed by the imaging means 103, and it does not comprise mechanical operating parts in its composition. Moreover, the optical characteristics selecting means 102B is situated in at least one light path of the separated light paths, and it selectively extracts the optical characteristics of at least one image of the separated plurality of images. For the optical characteristics selecting means 102B, it is possible to use, for example, a wavelength filter which selectively extracts or removes a particular wavelength component.

According to the optical system 102 shown in FIG. 8, the separating and image forming means 102A separates a single image 9 to be detected into a plurality of images, and each of the separated images is formed on a single imaging means 103, whereby it is possible to obtain a plurality of images (first image 103a, second image 103b) from a single object to be detected. Consequently, it is possible to eliminate disparity in measurement timing between respective images, and hence images can be compared in a case where there is time change in the object under investigation.

Furthermore, by introducing the optical characteristics selecting means 102B, it is possible to detect images having different optical characteristics, such as different wavelength, or the like. By selecting wavelength characteristics corresponding to oxygenated haemoglobin and deoxygenated haemoglobin as these optical characteristics, it is possible to use the system as a biological monitor, such as an oxygen monitor, or the like.

By means of the optical image measuring device according to the present embodiment, by using a single imaging element and dividing the imaging area thereof into a plurality of regions, it is possible to eliminate variation in imaging characteristics between imaging elements, which presents a problem when a plurality of imaging elements are used. It is also possible to suppress costs by reducing the number of imaging elements in this manner.

A further example of the optical image measuring device according to the present embodiment comprises: single imaging means; a combination of optical elements creating a plurality of light paths which form images in the same imaging direction by splitting a single object to be detected by means of single imaging means; and a wavelength selector element for selecting particular wavelength components, situated in at least one of the light paths. The single imaging means detects a plurality of images comprising different wavelength components obtained by splitting the single image to be detected.

The optical elements, composed of beam splitters using half-mirrors, prisms, or the like, or lens systems, and mirrors, or the like, form light paths having a uniform optical enlargement ratio. If they have different optical enlargement ratio, then it is necessary to perform image processing for matching the size of image formation, and further it is necessary to compensate for the effects caused by the difference in the size of pixels to be measured, in image evaluation.

For the reason mentioned above, calculation processing of each respective image can be readily performed by making uniform the characteristics, other than the desired optical characteristic, such as the wavelength.

The wavelength selecting element performs wavelength selection for removing a particular wavelength, or wavelength selection for transmitting a particular wavelength, or the like, thereby differentiating the wavelength components of the images formed on the imaging means. By setting the wavelength component selected by the wavelength selecting element in accordance with the item to be measured, it is possible to acquire data for the wavelength component required to make the desired measurement.

A plurality of images have the same imaging direction and the same observation direction in each of separated paths by using a single image to be detected, thereby providing an optical system which ensures uniformity between the plurality of images.

In a further example of an optical image measuring device according to the present embodiment, the imaging means is provided with image processing means for individually recognizing a plurality of images and performing image data processing. The image processing means is able to align positions between respective images, and to perform image evaluation, on the basis of markers contained in each respective image.

The marker comprises a positional alignment mark and an image evaluation mark. The positional alignment mark may be constituted by a cross sign, or the like, and a plurality of images formed on a single imaging means can be aligned in position on the image data, by using the positional alignment mark as a positional reference. Furthermore, the image evaluation mark comprises a mark corresponding to the optical characteristics selected by the optical characteristics selecting means, whereby evaluation by the optical characteristics selecting means, or detection of light source variation can be performed using the images detected by the imaging means. The image evaluation mark can be a coloured mark of the wavelength region selected by the wavelength selector elements. The marker may also be provided in the object under investigation or in the background section.

By means of the optical image measuring device according to this embodiment, correction between images is readily performed, and the accuracy and speed of data processing can be increased.

The present embodiment is now described further with respect to FIG. 9 to FIG. 14.

FIG. 9 to FIG. 13 are approximate diagrams for describing a first to fifth example of an optical image measuring device according to the present embodiment. The following description relates to examples wherein the optical image measuring device according to the present embodiment splits the image to be detected into two images, but the number of images into which it is split is not limited to two, and may be set to any desired plurality of images.

FIG. 9 shows a first example of an optical image measuring device according to the present embodiment. In FIG. 9, the image 9 to be detected of a measurement subject S is split into images 30a, 30b by the optical system 40, these images being formed on the imaging means 30. The optical system 40 comprises lenses 40A1, 40A6, a half mirror 40A2 and mirrors 40A3, 40A4, 40A5, a first light path 110a (indicated by the solid line) being constituted by the lens 40A1, half-mirror 40A2, mirror 40A3, 40A4 and lens 40A6, and a second light path 110b (indicated by the broken line) being constituted by the lens 40A1, half-mirror 40A2, mirror 40A5, and lens 40A6, thereby splitting the image 9 to be detected into two images 30a, 30b.

Each of the aforementioned optical elements is positioned in such a manner that that the first light path 110a and the second light path 110b have the same optical characteristics, such as optical enlargement ratio, polarization characteristics, and the like, with the exception of wavelength characteristics, and furthermore, in the imaging means 30, the images 30a and 30b have the same imaging direction to be obtained from the image 9 to be detected at the same timing or at approximately the same timing. Here, “approximately the same timing” comprises time differences wherein the time change in the measurement subject can be ignored.

As optical characteristics selecting means, for example, a wavelength filter 40B comprising a first wavelength filter element 40Ba and a second wavelength filter element 40Bb is inserted in an optically similar position in the first light path 110a and the second light path 110b. The first wavelength filter element 40Ba and the second wavelength filter element 40Bb are inserted respectively into the first light path 110a and the second light path 110b and perform processing such as extracting or removing a particular wavelength from the image 9 to be detected, thereby creating image 30a, 30b. It is also possible to adopt a composition wherein the wavelength filter 40B is only inserted into any one light path of the first light path 110a and second light path 110b, no filter being provided in the other light path.

The lens 40A1, half-mirror 40A2, and mirrors 40A3, 40A4, 40A5 split the image 9 to be detected of the measurement subject S into two, and these images are formed on the wavelength filters 40Ba, 40Bb at 1:1 magnification. The lens 40A6 forms the respective images created on the wavelength filters 40Ba, 40Bb, on the imaging surface of the imaging means 30. In so doing, the lens 40A6 forms the images in parallel in the same direction, without overlapping of the images. It is also possible to use a prism instead of a half-mirror.

Therefore, the images 30a, 30b formed on the imaging means 30 are images containing specific wavelength components, taken from the image 9 to be detected at the same timing or approximately the same timing. The wavelength selection properties of the wavelength filter 40B can be set according to the required measurement item, and its position in the light path can also be set as desired.

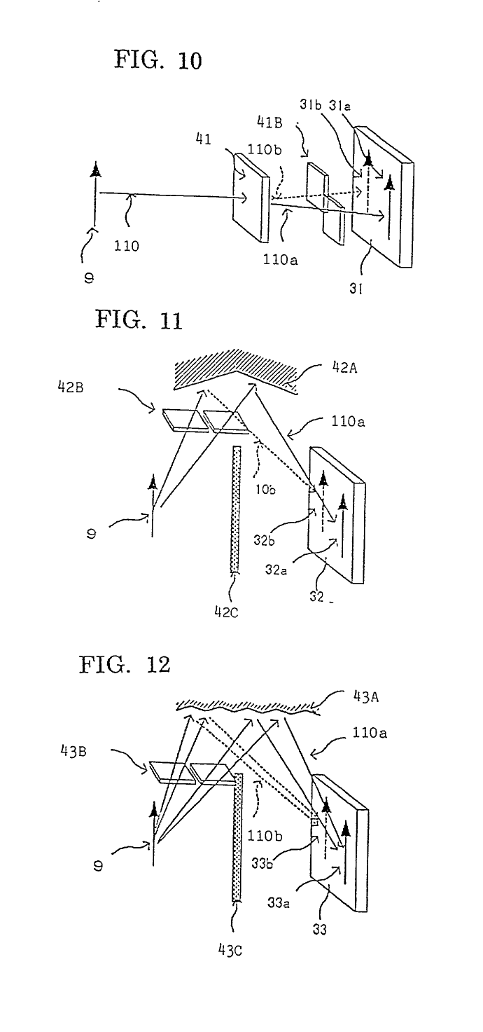

FIG. 10 shows a second example of an optical image measuring device according to the present embodiment. FIG. 10 is an example wherein a high-speed switching circuit is used as separating and image forming means. In FIG. 10, a high-speed switching circuit, such as a digital micro-mirror display 41, or the like, is positioned in the light path 110, whereby images are switched at high-speed within the same imaging operation, to form two images (images 31a, 31b) on the same imaging surface of the imaging means 31.

Since the high-speed switching circuit is able to perform a switching operation at sufficiently high speed compared to the rate of change of the measurement subject, it is possible to obtain images formed on the imaging surface which can be considered ones taken within the same imaging operation. Moreover, since the high-speed switching circuit has a simple structure, the composition of the optical image measuring device can be simplified. Incidentally, numeral 41B denotes a wavelength filter.

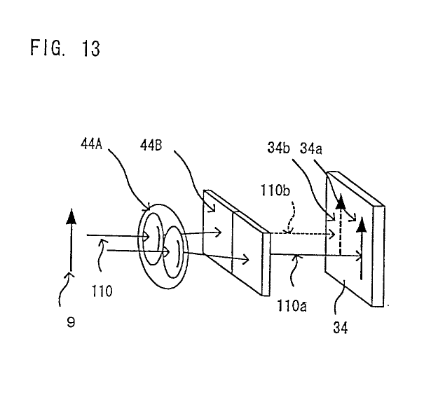

Furthermore, a third and fourth example of the optical image measuring device according to the present embodiment are illustrated in FIG. 11 and FIG. 12. These third and fourth examples use mirrors as the separating and image forming means.

In the third example shown in FIG. 11, the image 9 to be detected is split using two mirrors 42A, whereby two images (image 32a, 32b) are formed on the same imaging surface of the imaging means 32. Moreover, in the fourth example shown in FIG. 12, the image 9 to be detected is split using a multi-mirror 43A comprising a plurality of reflective surfaces, whereby two images (image 33a, 33b) are formed on the same imaging surface of the imaging means 33.

In FIG. 11 and FIG. 12, the members indicated by numerals 42C and 43C are shielding members situated in such a manner that an images of the image 9 to be detected is not formed directly on the imaging means 32, 33, by means of which it is possible to form an image on the imaging means 32, 33 by means of the mirror 42A or multi-mirror 43A only. Furthermore, for the multi-mirror used in the third example, it is also possible to use, for instance, a coarse diffraction lattice having a spacing of the order of 1 line/mm. Moreover, numerals 42B, 43B show wavelength filters.

A fifth example of an optical image measuring device according to the present embodiment is shown in FIG. 13. This fifth example uses a lens system as the separating and image forming means. In FIG. 13, a compound lens or multiple-focus lens system 44A is positioned in the light path 110, whereby the image 9 to be detected is split and two images (image 34a, 34b) are formed on the same imaging surface of the imaging means 34. The lens system 44A for splitting the image 9 to be detected may also be constituted by using a combination of simple lenses, or a holographic lens, or the like, instead of the compound or multiple-focus lens system. Numeral 44B indicates a wavelength filter.

Next, the image data processing performed by the optical image measuring device according to the present embodiment will be described. The image data captured by the imaging means 103 is processed by the image processing means which recognizes a plurality of images individually. This image processing means is able to align the respective positions of the images and to perform image evaluation, on the basis of markers contained in each image.

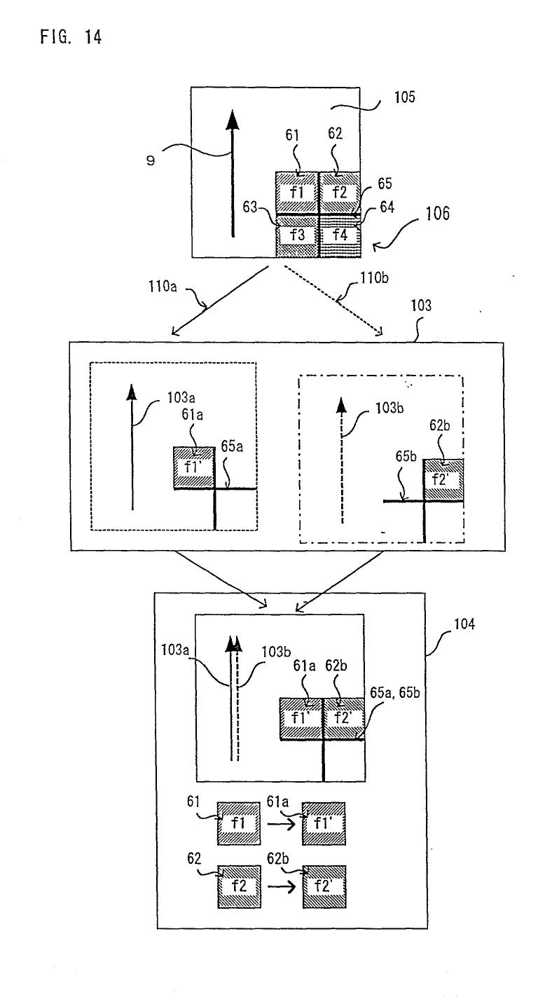

FIG. 14 is a diagram for describing image processing using a marker. A marker 106 comprises a cross line 65 for positional alignment, and marks 61 to 64 for image evaluation, and it is provided either in the image 9 to be detected or in a background section 105. FIG. 14 shows an example wherein the marker 106 is provided in the background section 105.

Since the images on the imaging means 103 are formed in such a manner that they do not overlap on the imaging surface, it is difficult to perform positional alignment of the images using the image data alone. Therefore, a positional alignment cross line 65 is used to align their respective positions. The cross lines 65a, 65b are formed in similar positional relationships with respect to the images 103a, 103b, on the imaging means 103. Therefore, in the image data processing performed by the image processing means 104, it is possible to align the positions of the image 103a and the image 103b by taking the cross lines 65a, 65b as reference points for registering position.

Moreover, the marks 61 to 64 for image evaluation are marks corresponding to the optical characteristic selected by the optical characteristics selecting means situated in the light path, and they may use, for example, colours corresponding to the wavelength regions fl to f4 of a wavelength filter.

The image 103a is an example of an image that has been transmitted through a wavelength filter of wavelength region f1, a mark 61a after transmission being formed thereon together with the cross line 65a. Furthermore, the image 103b is an example of an image that has been transmitted through a wavelength filter of wavelength region f2, a mark 61b after transmission being formed thereon together with the cross line 65b.

By comparing the wavelength characteristics f1′, f2′ of the marks 61a, 62b formed on the imaging means 103, and the wavelength characteristics f1, f2 of the marks 61, 62, it is possible to evaluate the properties of the optical characteristics selecting means, such as wavelength filters, or to detect spectral variation in the light source. Moreover, this comparison allows to inspect the light path along which the formed image has been transmitted, thus functioning as data marking during data processing.

According to the various examples described above, benefits of the following kind are obtained.

Since the optical measuring device requires a single imaging element, the composition of the optical image measuring device can be simplified and reduced in cost.

Since the measurement timings for a plurality of wavelengths can be set to effectively the same timing, it can be assumed that this is no disparity in measurement timings, and hence it is possible to obtain a plurality of image data for time change in a measurement subject experiencing rapid change.

By reducing the mechanical drive sections, it is possible to improve the reliability of the optical image measuring device.

With the optical characteristics selecting means, the combination of optical characteristics, such as wavelength, being measured simultaneously and in parallel, can be changed readily.

Moreover, according to the first example of the present embodiment, since an image to be detected coming from the same direction is split into a plurality of images, which are formed in the same imaging direction, it is possible to suppress distortion between the captured images, even when the image to be detected is enlarged.

According to the foregoing description, by means of an optical image measuring device according to the present embodiment, it is possible to ensure measurement synchronism by measuring a plurality of image having different optical characteristics, such as different wavelengths, without generating difference in the respective measurement timings thereof, and furthermore, as it is possible to obtain a plurality of images at high-speed, it is possible to detect a time change in a measurement subject experiencing rapid change and then it is possible to obtain a plurality of images having different optical characteristics.

Next, a third embodiment of an optical biological measurement method according to the present invention and a device for implementing said method are described with reference to FIG. 20 to FIG. 25.

FIG. 20 shows an approximate view of an optical biological measurement device. In FIG. 20, optical detecting means 20, such as a two-dimensional detector, or the like, detects the light emitted by the measurement subject 10unk and light emitted by a reference subject 10std having time independent reflectivity, when irradiated by a light source 11, measurement image data D1(unk), D2(unk), . . . , Dn(unk) being determined for a plurality of wavelengths (wavelength λ1 to wavelength λn) with respect to the measurement subject 10unk, and reference image data D1(std), D2(std), . . . , Dn(std) being determined for the same plurality of wavelengths (wavelength λ1 to wavelength λn) with respect to the measurement subject 10std. The reference subject 10std having time independent reflectivity is desired to have uniform reflectivity, but it is correctable if its local variation is known and correctable after measurement. The value of its reflectivity can be set to any desired reflectivity within the measurement range of the two-dimensional detector, for example, white or grey card may be used. Taking into account the ratio between the detection noise level and the detection signal level, it is desirable that the reflectivity of the reference subject should be of the same order as that of the measurement subject.

Here, the measurement data D1(unk), D2(unk), . . . , Dn(unk) are not single values, but rather represent a pixel array. For example, in the case of a 100×100 image having 10,000 pixels, D1(unk) will essentially have 10,000 values for each pixel of the 100×100 pixel array. Here, in order to simplify the notation, D1(unk) is taken to represent one pixel of the pixel array. Therefore, where D1(unk), D2(unk), . . . , Dn(unk) appear in the calculation, this signifies that there are similar values as many as total number of pixels in the array.

Moreover, the reference image data D1(std), D2(std), Dn(std) represents data for a pixel unit, rather than a single value. The reference image data may comprise data for the same number of pixels as the total number of responding pixels of the measurement image data, or may comprise one data item for a certain plurality of pixels of the measurement image data, or may comprise one reference image data for all the pixels of the measurement image data. The reference image data may be determined before or after measurement of the measurement subject, or it may be set in advance.

In image calculation for deriving two-dimensional information for absolute values relating to body tissue, calculation for applying prescribed weightings different for every measurement wavelength, to the measurement image data and the reference image data, and then adding the results, is performed for every pixel unit.

Calculation f is one example of image calculation, wherein prescribed weightings are applied for each different wavelength, to the differences between the measurement image data and the reference image data, using the measurement image data D1(unk) to Dm(unk) and the reference image data D1(std) to Dm(std) of wavelength λ1 to λm, taken from the image data for a plurality of wavelengths λ1 to λn, and then the results are added, so that biological information is obtained from the calculation. The details of the calculation and the weightings used in calculation correspond to the biological information to be derived and to the wavelength. If the reference image data D1(std) to Dm(std) is only one-pixel data for each wavelength, then calculation is performed using one data for each wavelength, with respect to each pixel data of the measurement image data D1(unk) to Dm(unk).

The present invention performs image calculation with respect to reference image data obtained by measuring a reference body of time independent reflectivity and measurement image data obtained from a measurement subject, and thereby, even if the measurement image data changes due to variation in the optical measurement conditions under which the subject is measured optically, for example, variation in the intensity of the light source, the distance between the light source and the subject, or the distance between the subject and the two-dimensional detector, then such variation in the measurement conditions is offset by means of the reference image data receiving the same variation of measurement conditions, and hence absolute values at the measurement time point can be derived.

In the aforementioned description, a linear relation is assumed between the measured absorbance and the absorption coefficient that is defined by the concentration of hemoglobin (Hb). The assumption is more correct when a change in Hb concentration is smaller. In most cases the above assumption is approximately valid, however, it is found a non-linear relation would give more accurate results according to Monte Carlo simulation. This paragraph emphasizes that the proposed method can be easily extended to the non-linear case.

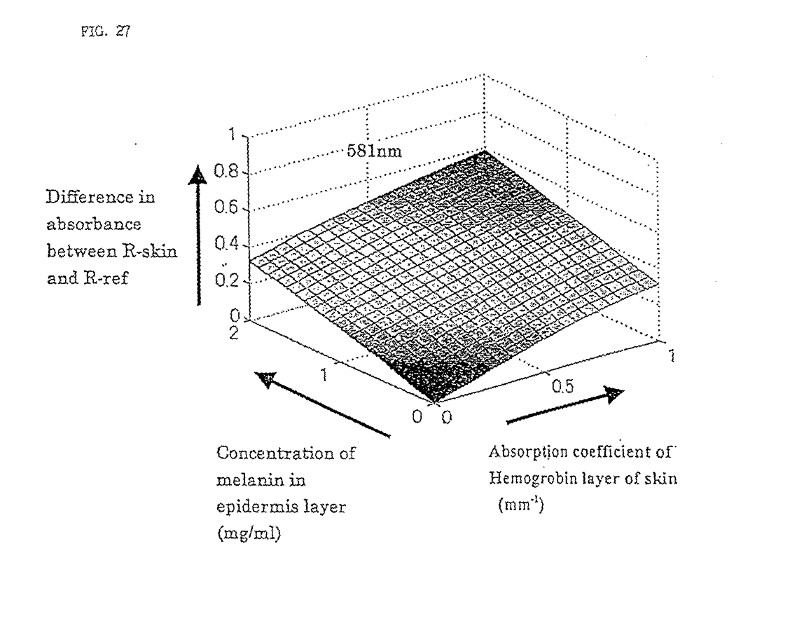

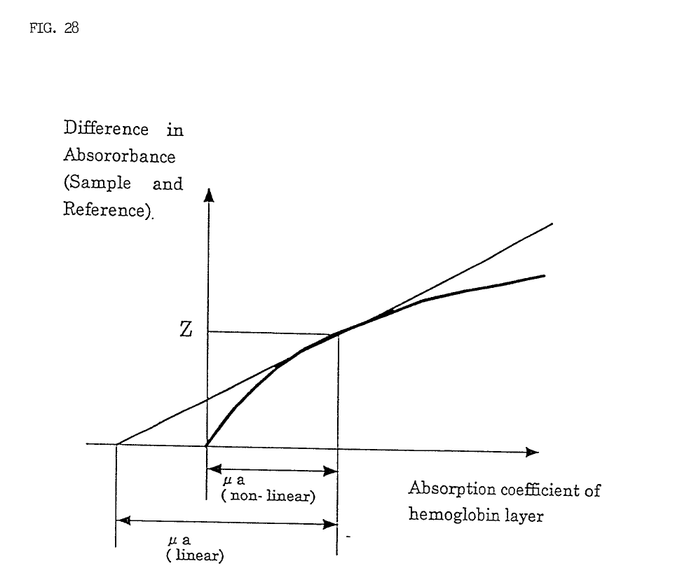

FIG. 27 shows an example of Monte Carlo simulation that is carried out for a skin model consisting of 0.06 mm thick epidermis layer and 1 mm thick dermis layer, assuming that the epidermis contains melanin and dermis contains oxyHb and deoxyHb. The graph is three dimensional, x-axis being absorption coefficient of the dermis layer that includes Hb, y-axis being melanin concentration and z-axis being the difference in absorbance between a sample and the reference with no absorption.

The absorption coefficient μa (x axis in the FIG. 27) of the dermis layer is defined by

μa=1n10·{ε_{oxy}[oxyHb]+ε_{deoxy}[deoxyHb]} (5′)

where ε_{oxy} and ε_{deoxy} are the molar absorptivity for oxyhb and deoxyHb respectively and [oxyHb] and [deoxyHb] are the concentration of oxyHb and deoxyHb included in the dermis layer. The factor (ln 10) is required to adjust the ratio of natural logarithm and ordinary logarithm because μa is traditionally based on the natural logarithm while ε_{oxy} and deoxy are usually based on the ordinary logarithm.

FIG. 28 makes clear the relation between linear and nonliner case. The x-axis (μa) and z-axis (Z=difference in absorbance) are the same as the FIG. 27. The y-axis is missing because the FIG. 28 is drawn at a particular melanin concentration. It has a non-linear curve and a linear curve, the latter being considered a tangent at a particular point on the non-linear curve.

When a measured Z value is given by a measurement at a wavelength, corresponding μa value is determined by the nonlinear curve. Therefore if we make measurements at plural wavelengths, we have many μa values appearing in equation (5′). Then equation (5′) for plural wavelengths becomes a simultaneous equation which determines [oxyHb] and [deoxyhb]. The μa value is now considered a kind of “measured” value. Note here a non-linear process exists only at the conversion from Z to μa. Once μa's are obtained for plural wavelengths, the later process is to solve the linear simultaneous equation (5′), because the converted μa values are already linear with respect to the concentration of such as hemoglobin. This means when a value of measured absorbance is given, converted value μa is considered as a measured “quasi-absorbance”. So the argument made with the linear case, until now and from now on, can be extended to the non-linear case using this process. For example in the previous equation (1), introduction of nonlinear process could be attained if we substitute D1, D2 and etc by corresponding quasi-absorbance D1′, D2′ and etc. Also in the equation (7) appearing in the later description, it is enough to use converted “quasi-absorbances” Dg(std)′, Dg(unk)′, Dr(std)′ and Dr (unk)′, in place of Dg(std), Dg(unk), Dr(std) and Dr(unk).

Another important points included in FIG. 28 is a comparison of the results with non-linear and linear case. The conversion from Z to μa, can be carried out in both non-linear case and linear case. The results, μa (non-linear) and μa(linear), are shown in the figure. Because the tangent crosses the x-axis at the left side of the origin, μa (linear) tends to be larger than the correct μa (non-linear). This fact indicates that the result with linear method is less accurate than that of non-linear method, the former is still useful because it is qualitatively right and simpler.

The reference image data can be determined in a variety of modes, as described below.

The reference image data according to a first mode is obtained, independently of the biological measurement, by measuring a plurality of pixels of a reference subject of time independent reflectivity, at a plurality of measurement wavelengths. The reference image data according to this mode comprises data corresponding to each pixel of the measurement image data, and two-dimensional information for absolute values can be derived by calculating with respect to the measurement image data and the reference image data for the corresponding pixel. In this first mode, it is possible to offset variation in data values due to the distance between the light source and the two-dimensional detector by ensuring the same optical measurement conditions for the reference subject and the measurement subject.

The reference image data according to the second mode is derived by placing a single reference subject having time independent reflectivity on the body and detecting the image data at one location thereon at a plurality of measurement wavelengths, whereupon a single reference image data is obtained from the image data for a single pixel, or image data calculated from a plurality of pixels. The reference image data according to this mode is a single reference image data, and two-dimensional information for absolute values can be derived by calculating with respect to the respective pixels of the measurement image data and this single reference image data.

The reference image data according to the third mode is two-dimensional reference image data derived by placing a plurality of reference subjects of time independent reflectivity on the body, detecting image data at a plurality of location for a plurality of measurement wavelengths, and the interpolating with respect to the image data for the plurality of locations. The reference image data according to this mode is two-dimensional reference image data, and is derived by calculating with respect to the measurement image data and the reference image data for the corresponding pixel.

The reference image data according to the fourth mode is derived as single reference image data from the image data detected using a plurality of measurement wavelengths at a location of the body producing little time change. The reference image data according to this mode is a single reference image data, and two-dimensional information for absolute values is derived by calculating with respect to each pixel of the measurement image data and the single reference image data.

In the present invention, the measurement wavelengths are taken as at least two wavelengths between 400 nm and 1000 nm, and the amount of oxy-haemoglobin or the amount of deoxy-haemoglobin is determined using the measurement image data and reference image data derived at the aforementioned two wavelengths.

As shown in equation (6) below, the absolute amount of oxy-haemoglobin [OxyHb] or the absolute amount of deoxy-haemoglobin [deOxyHb] is determined by applying prescribed weightings k1, k2, k3, k4 to the values of respective pixels in four image data obtained from measurement image data D1(unk), D2(unk) of the reference image data at wavelengths λ1, λ2 and reference image data D1(std), D2(std), and then adding or subtracting, appropriately.

[OxyHb]=k1×D1(unk)+k2×D2(unk)−k1×D1(std)−k2×D2(std)

[deOxyHb]=k3×D1(unk)+k4×D2(unk)−k3×D1(std)−k4×D2(std) (6)

D1(unk) and D2(unk) indicate values representing one pixel of the measurement image data at wavelengths λ1, λ2, and D1(std) and D2(std) indicate values representing one pixel of the reference image data at wavelengths λ1, λ2. The quantities of D1, D2, and the like, described above are either the actual outputs of the two-dimensional detector for each image, or values obtained by subtracting a dark signal, or values obtained by logarithmic conversion.

In equation (6) above, [OxyHb] is the pixel value of the absolute value of the oxy-haemoglobin, and [deOxyHb] is the pixel value of the absolute value of the deoxy-haemoglobin. The value of [OxyHb] is obtained by summing the four pixel values D1(std), D2(std), D1(unk), D2(unk) obtained from the two wavelength components of the measurement image data and reference image data, after respective weightings (−k1, −k2, k1, k2) have been applied to each of the values. Moreover, the value of [deOxyHb] is obtained by summing the four pixel values D1(std), D2(std), D1(unk), D2(unk) obtained from the same source, after respective weightings (−k3, −k4, k3, k4) have been applied to each of the values. Since the aforementioned calculations are applied to each pixel, the obtained image is an image wherein each pixel shows the absolute value of the oxy-haemoglobin or deoxy-haemoglobin.

By converting the derived two-dimensional information relating to the organism into an image, it is possible to confirm the biological information visually. For example, it is possible to confirm the state of the oxy-haemoglobin or the deoxy-haemoglobin at the measurement time point, by means of an image, at a location in the blood vessel distribution where there is a blood vessel blockage, or the like, and hence biological situations, such as a blood vessel blockage, can be diagnosed.

According to the present invention, since the absolute values at the measurement time can be obtained, there is no need to cause the state of the blood flow in the body to change, as in prior art diagnosis involving measurement of relative values, and hence operations such as tightening by means of a cuff, or the like, become obsolete.

Moreover, a further embodiment of an optical biological measurement method according to the present invention creates an external image on the basis of one image data of the multiple-wavelength image data measured by the optical biological measurement according to the present invention and displays this external image in superimposed fashion with image derived from the multiple-wavelength image data. By means of this display, the position of the biological information can be confirmed readily. In so doing, since only one of the original multiple-wavelength images is used to create the external image, a major benefit is obtained in that an external image and functional images can be mutually superimposed, without requiring an extra hardware device.

According to the optical biological measurement device of the present invention, it is possible to display an image of the activity distribution of oxygen supply to various parts of body tissue, and hence abnormalities, such as blockages in blood vessels, or the like, can be identified from the resulting patterns. Moreover, according to the present invention, since neither X rays nor image forming agents are required, it is possible to reduce the effects on the body, and hence it is possible to achieve a measuring device suitable for repeated measurement, for example, in judging clinical effects.

By means of the optical image measuring device according to the present embodiment, it is possible to obtain two-dimensional image data by adopting a two-dimensional detector, and hence a plurality of light transmitters and receivers are not required and the device can be compactified, in addition to which the price of the device can be reduced and furthermore, since no medicines are used in measurement, the cost of investigation can be reduced.

Moreover, in the optical biological measurement method according to the present invention, it is possible to apply the following method for removing fluctuating components contained in the detection signal which are not dependent on wavelength.

Upon evaluating the optical biological measurement, the inventors of the present invention found that the detection signal contains wavelength-independent fluctuations, and further discovered a method for removing these fluctuations.

Whereas the reference subject is usually flat, the measurement subject is generally three-dimensional. Therefore, the portions which are oblique to the light source, for instance, will be in shadow, and the intensity of the light irradiated thereon will be weaker. Consequently, the illumination conditions for the shadow region of the measurement subject will be different to those for the flat face of the reference subject. This means that when calculation is made using the same coefficients in equation (6) above, a significant error will arise in the shadow region. The fluctuation in the detection signal caused by the indentations in the measurement subject are wavelength-independent components.

Therefore, in order to remove these wavelength-independent variations, by adding a variable for the wavelength-independent fluctuation to the variables of oxy-haemoglobin and deoxy-haemoglobin, and resolving a simultaneous equation using a total of three variables, then the wavelength-independent variation can be removed from the oxy-haemoglobin and deoxy-haemoglobin. Since this simultaneous equation comprises three variables, then it is necessary to have at least three measurement wavelengths in order to resolve the simultaneous equations.

Below, this is explained in the case of three wavelengths. Setting the three variables as X for oxy-haemoglobin, Y for deoxy-haemoglobin and E for the wavelength-independent variation, the following simultaneous equation is established:

Wavelength 1: λ1εoxyλ1·X+εdeoλ1·Y+E=ΔABSλ1

Wavelength 2: λ2εoxyλ2·X+εdeoλ2·Y+E=ΔABSλ2

Wavelength 3: λ3εoxyλ3·X+εdeoλ3·Y+E=ΔABSλ3

Here, X=[OxyHb], Y=[deOxyHb], E=wavelength-independent light absorption element; and ΔABS indicates the difference between the body image and the reference image.

By resolving the simultaneous equation, the oxy-haemoglobin (X) and deoxy-haemoglobin (Y) in the following equation (6′) are obtained as solutions.

X=k1·ΔABSλ1+k2·ΔABSλ2+k3·ΔABSλ3

Y=k4·ΔABSλ1+k5·ΔABSλ2+k6·αABSλ3

E=k7·ΔABSλ1+k8·ΔABSλ2+k9·ΔABSλ3

Here, ΔABSλ1 is ΔABSα1=−log (signal obtained from measurement subject after dark signal correction/signal obtained from reference surface after dark signal correction).

Equation (6′) has the same form as equation (6), except for the fact that one extra wavelength is required.

Moreover, in the case of a system which can only measure two wavelengths, it is possible to apply the aforementioned method for removing the wavelength-independent fluctuation with respect to the difference signal, by reducing the variables by restricting the calculation to a difference signal which contains both oxy-haemoglobin and deoxy-haemoglobin simultaneously, after the correction signal of E has been used.

By means of the aforementioned method, it is also possible to reduce the error arising in shadowed regions of the measurement subject, which is peculiar to an absolute value measurement.

An example of a device for implementing the optical biological measurement method according to the present invention is now described with reference to FIG. 21. FIG. 21 shows an example of two-wavelength measurement. The optical biological measurement device 200 comprises image measuring means 2, image calculating means 3, and image display processing means 4 for displaying the obtained image.

The image measuring means 2 comprises optical detecting means 20 for detecting light emitted from a measurement subject 10unk and a reference subject 10std due to illumination by a light source 11, by means of a two-dimensional detector, or the like, and outputting two-wavelength (wavelength λg and wavelength λr) image data; and image data acquiring means 27 for acquiring the aforementioned image data. The intensity of the light source 11 and the reflectivity of the reference subject 10std are selected in such a manner that the intensity of the emitted light comes within the measurement range of the two-dimensional detector. For the reference subject 10std having time-independent reflectivity, it is possible to use a white or grey member.

According to the present invention, when obtaining the amount of oxy-haemoglobin or the amount of deoxy-haemoglobin, at least two wavelengths between 400 nm and 1000 nm are taken as measurement wavelengths, and image calculation is performed using the measurement image data D(unk) and reference image data D(std) derived at these two wavelengths.

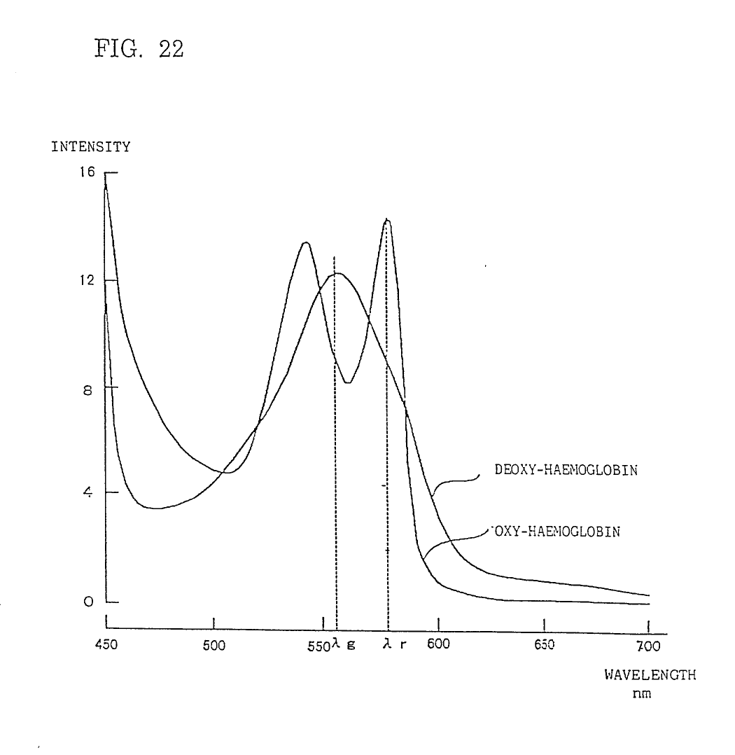

FIG. 22 is an approximate view of the wavelength spectra of oxy-haemoglobin and deoxy-haemoglobin. Of the peak points appearing in the wavelength spectra in FIG. 22, for example, 554 nm which is one peak point for oxy-haemoglobin is taken as the measurement wavelength λg, and 578 nm which is one peak point for deoxy-haemoglobin is taken as the measurement wavelength λr. The measurement wavelengths are not limited to these wavelength values, and may be set to other wavelength values.

The image data acquiring means 27 acquires reference image data Dg(std) Dr(std) at wavelength λg and wavelength λr from the reference subject 10std, and measurement image data Dg(unk) and Dr(unk) at wavelength λg and wavelength λr from the measurement subject 10unk. The image calculating means 3 performs calcuational processing using these four types of image data. Here, the image data Dg(std), Dr(std), Dg(unk), Dr(unk) are not single values, but rather, they represent an array of pixels.

Here, the signal values for the respective pixels represent each pixel of the CCD; for instance, in a 512×600 pixel CCD, each of the approximately 300,000 pixels will have a respective signal value corresponding to the received image. Moreover, the respective sizes of the pixel signal value Dg at wavelength λg and the pixel signal value Dr at wavelength λr are integers in the range of 0<=(Dg, Dr)<=4095(=2¹²), in the case of a 12-bit signal; and when the values of Dg, Dr are small, then this indicates weak light intensity, and when the values of Dg, Dr are large, this indicates strong light intensity.

The image calculating means 3 obtains the pixel value of the absolute quantity of oxy-haemoglobin [OxyHb] and the pixel value of the absolute quantity of deoxy-haemoglobin [deOxyHb] by performing a calculation according to the following equation (7) wherein Dl equation (6) is substituted by Dg and D2 is substituted by Dr:

[OxyHb]=−k1(log(Dg(std)/Dg(unk)))−k2(log(Dr(std)/Dr(unk)))

[deOxyHb]=−k3(log(Dg(std)/Dg(unk)))−k4(log(Dr(std)/Dr(unk))) (7)

The image display processing means 4 converts the absolute quantities for oxy-haemoglobin [OxyHb] and deoxy-haemoglobin [deOxyHb] obtained by the image calculating means 3. Here, Dg and Dr are generally valves where a dark signal has been removed.

When the measurement wavelength λg is taken as 554 nm and the measurement wavelength λr is taken as 578 nm, then the coefficients k1, k2, k3, k4 respectively take values of 89.7, −124.5, −143.3 and 94.9, as shown in the following equations.

[OxyHb]=−89.7(log(Dg(std)/Dg(unk)))+124.5(log(Dr(std)/Dr(unk)))

[deOxyHb]=−143.3(log(Dg(std)/Dg(unk)))−94.9(log(Dr(std)/Dr(unk)))

Dg(std), Dr(std) are reference images at 554 nm and 578 nm, and Dg(unk) and Dr(unk) are body images at 554 nm and 578 nm.

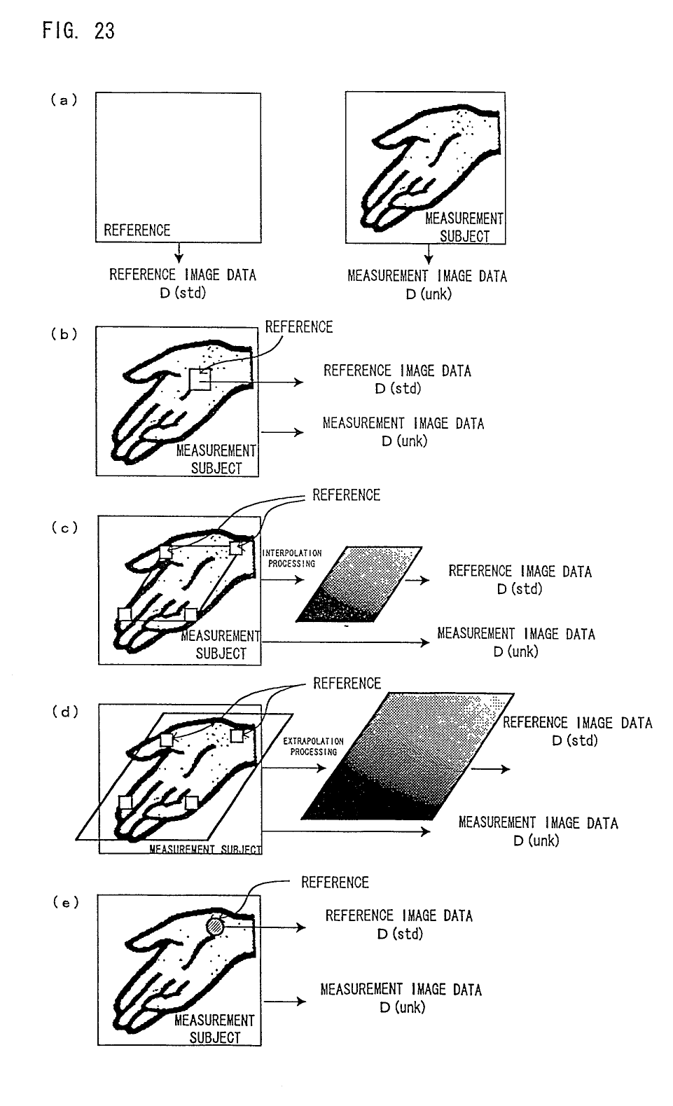

There follows a description of an example wherein reference image data is obtained using FIG. 23.

FIG. 23(a) is an example wherein a reference subject having time independent reflectivity is obtained at a plurality of measurement wavelengths by means of a plurality of pixels, independently of biological measurement. In this example, a reference subject of approximately the same size as the measurement subject is prepared, image data is derived under the same optical measurement conditions, such as light source intensity, positional relationship between light source and two-dimensional detector, and the like, as the optical measurement conditions of the measurement subject, and the image data thus obtained is taken as reference image data D(std). The measurement image data obtained by measuring the measurement subject is taken as D(unk).

FIG. 23(b) is an example wherein a single reference subject of time independent reflectivity is placed over the measurement subject, and image data at a single location is detected at a plurality of measurement wavelengths. In this example, a small piece of time independent reflectivity is prepared and placed on the measurement subject, and image data for the reference subject is derived under the same optical measurement conditions as the optical measurement conditions of the measurement subject, this image data being taken as reference image data D(std).

FIGS. 23(c) and 23(d) show an example wherein a plurality of reference subjects of time independent reflectivity are placed on the measurement subject and image data is detected at the plurality of locations using a plurality of measurement wavelengths. In this example, a plurality of small pieces of time independent reflectivity are prepared and placed at prescribed intervals on the measurement subject, image data for these reference subjects is derived under the same optical measurement conditions as for the measurement subject, and two-dimensional reference image data is obtained by interpolation or extrapolation, using the plurality of image data thus derived, and set as reference image data D(std).

FIG. 23(e) is an example wherein image data detected using a plurality of measurement wavelengths in a location on the measurement subject experiencing little time change is obtained as a single reference image data. In general, it has been known that there are some regions of the body which have a little time change in blood flow. This example uses a location of this kind which displays little change as a reference, taking a portion of the measurement data for the measurement subject as reference image data D(std).

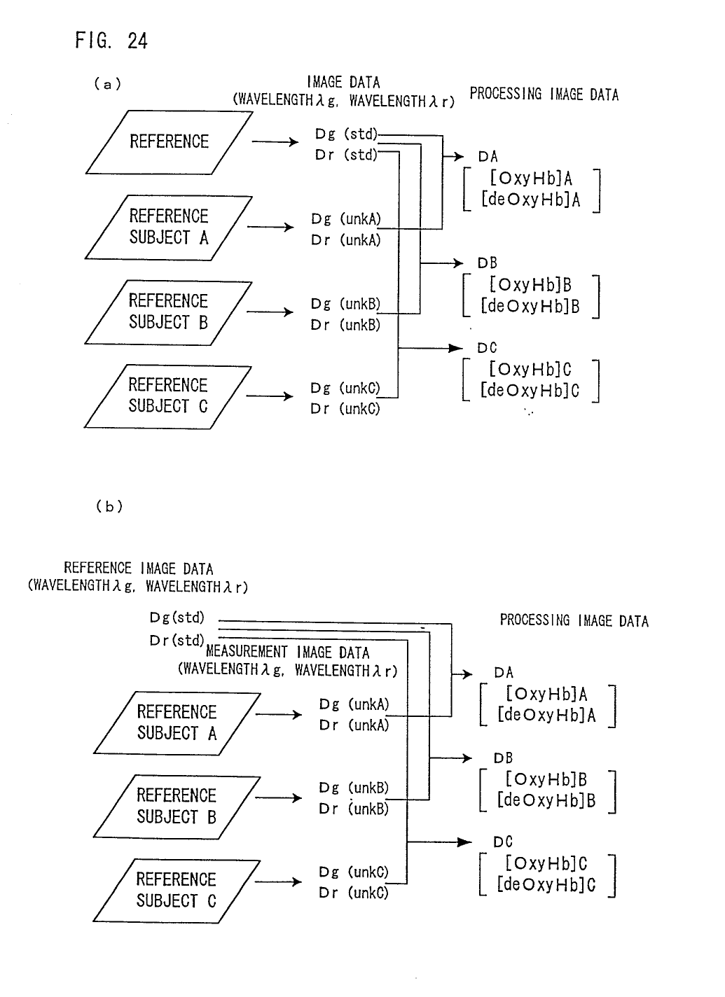

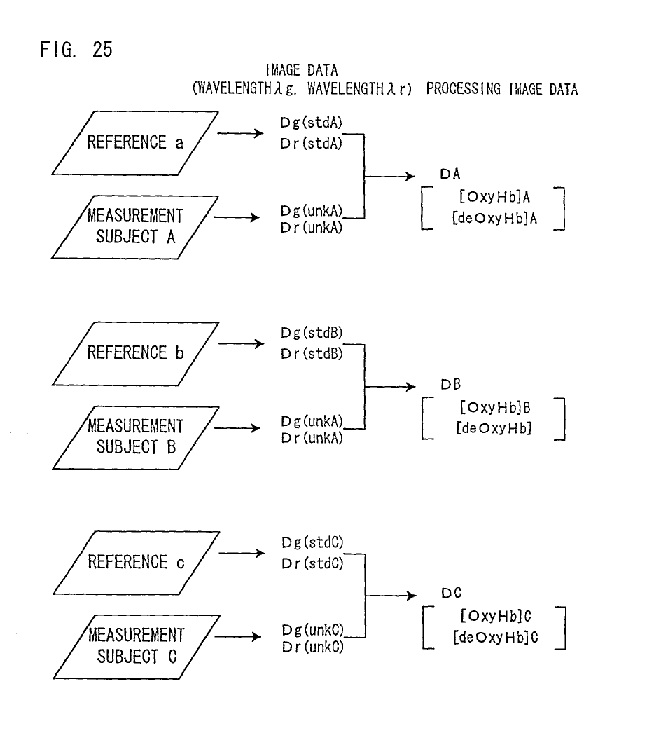

In the measurement according to the present invention, it is possible to adopt various combinations with regard to reference image data and measurement image data. Below, combinations of reference image data and measurement image data are described with reference to FIG. 24 and FIG. 25. The cases shown in FIG. 24 and FIG. 25 are for two-wavelength measurement.

FIGS. 24(a) and 24(b) are examples using one reference subject with respect to a plurality of measurement subjects. In FIG. 24(a), one reference subject is prepared for the measurement subjects A, B, C, and the reference image data Dg(std), Dr(std) determined from the one reference subject is used commonly with respect to measurement image data for the respective measurement subjects A, B, C, namely, Dg(unkA), Dr(unkA), Dg(unkB), Dr(unkB), Dg(unkC), Dr(unkC), to obtain DA, DB, DC. Furthermore, FIG. 24(b) is an example wherein reference image data Dg(std), Dr(std) are prepared in advance.

The examples in FIG. 24(a) and 24(b) can be applied to the measurement of a plurality of measurement subjects, in cases where there is little variation in the optical measurement conditions, and the processing for determining reference image data can be performed once or it can be omitted. This mode is suitable when measuring a large number of subjects in a short period of time, for instance, in a group diagnosis.

FIG. 25 is an example where reference subjects are used respectively for each measurement subject. In FIG. 25, respective reference subjects a, b, c are prepared for measurement subjects A, B, C, and the reference image data Dg(stda), Dr(stda) obtained from reference subject a is combined with the measurement image data Dg(unkA), Dr(unkA) obtained from measurement subject A, the reference image data Dg(stdb), Dr(stdb) obtained from reference subject b is combined with the measurement image data Dg(unkB), Dr(unkB) obtained from measurement subject B, and the reference image data Dg(stdc), Dr(stdc) obtained from reference subject c is combined with the measurement image data Dg(unkC), Dr(unkC) obtained from measurement subject C, thereby deriving DA, DB, DC. The example in FIG. 25 can be applied to measurement of a plurality of measurement subjects, in cases where there is significant variation in optical measurement conditions, this optical variation being offset by obtaining reference image data for each measurement operation.

To perform two-wavelength measurement by means of optical detecting means 20 as shown in FIG. 20 and FIG. 21, optical detecting means 20 such as the example illustrated previously in FIG. 2 (using a plurality of optical systems) or FIG. 3 (switching a single optical system) is used.

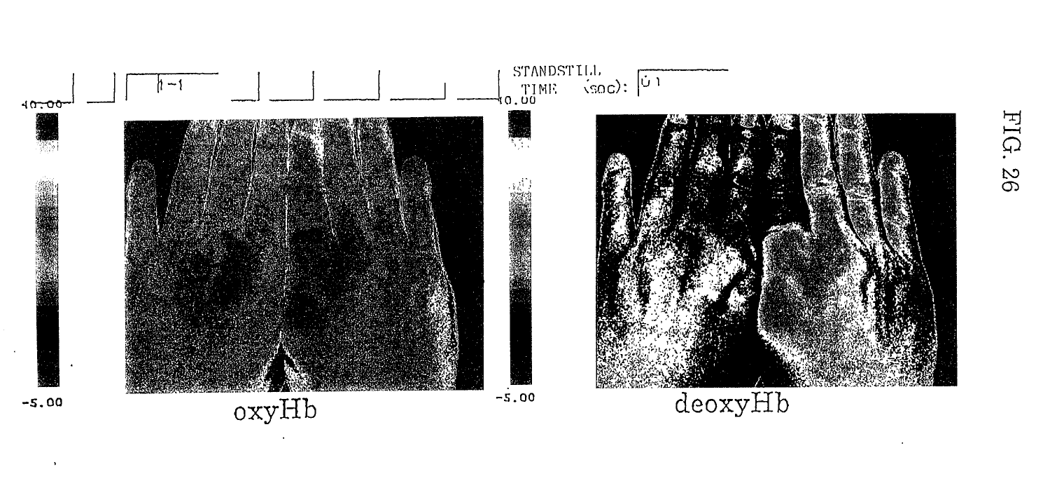

Next, FIG. 26 illustrates an example where absolute values for oxy-haemoglobin and deoxy-haemoglobin obtained by means of the optical biological measurement method according to the present invention are displayed as images.

In FIG. 26, the image on the right-hand side is an image indicating the quantity of oxy-haemoglobin and the image on the left-hand side is an image indicating the quantity of deoxy-haemoglobin. In both cases, the left hand is in a state where blood circulation has been cut off for two minutes by a pressure of 200 mmHg, and the right hand is in a state where blood circulation has been cut off by clamping the index finger. According to these image examples, in the image showing the amount of deoxy-haemoglobin, it is possible clearly to distinguish the parts of the body where blood circulation has been cut off and parts of the body where blood circulation has not been cut off. Therefore, by means of the optical biological measurement according to the present invention, it is possible to display the activity distribution of oxygen supply to various parts of body tissue, and hence abnormalities, such as blockages of blood vessels, or the like, can be identified.

Moreover, by means of the optical biological measurement method according to the present invention, it is possible to obtain an external image of the measurement subject using one wavelength output of the multiple-wavelength output from a two-dimensional detector, and therefore it is possible readily to verify the state of blood circulation, by superimposing the external image with the quantity of oxy-haemoglobin and the quantity of deoxy-haemoglobin, or the like. For example, it is possible to superimpose a display of an external image based on a highly contrasting black and white image, with a display of variation in the oxygenation status based on artificial colours. It is also possible to use an external image by removing the filter, or to use an image obtained when the filter is installed.

As described above, by means of optical biological measurement according to the present invention, absolute values at the measurement time can be obtained readily and in a short period of time, and the state of body tissue can be diagnosed, by means of two-dimensional image information for the measurement subject obtained by a single two-dimensional detector, without requiring a plurality of detectors.

Furthermore, by also combining correction for variation that is independent of the light wavelength, it is possible to reduce error caused by shadow effects, even if the illumination is shaded by the measurement subject.

What is claimed is:

1. An optical image measuring device comprising:
image measuring means for measuring image data of a plurality of wavelengths, over time; and
image calculating means for performing image calculation for determining biological information, using a plurality of image data having respectively different measurement wavelengths and measurement timings;
wherein light is irradiated onto an organism, and the light emitted from the organism is detected by a two-dimensional detector.

2. The optical image measuring device according to claim 1, wherein said image measuring means measures image data of at least two wavelengths, and said image calculating means obtains values relating to biological information by performing calculations for applying prescribed weightings to the respective image data or corresponding non-linearly converted image data of at least four images obtained from image data at least two timings, and then summing the results.

3. The optical image measuring device according to claim 2, wherein the values relating to biological information is the quantity of oxy-haemoglobin or the quantity of deoxy-haemoglobin.

4. The optical image measuring device according to claim 1, wherein said image calculating means comprises means for calculating images of time change at regular intervals with respect to time differential images or differences between images obtained at adjacent measurement timings.

5. The optical image measuring device according to claim 1, comprising inducing means for inducing a change in the state of the organism, thereby changing the light emitted from the organism.

6. An optical image measuring device comprising:
an optical system including separating and image forming means and optical characteristics selecting means; and
single imaging means for capturing a plurality of images obtained by means of said optical system, simultaneously or virtually simultaneously;
wherein said separating and image forming means separates a single image to be detected into a plurality of images which are formed on a single imaging means;
said optical characteristics selecting means selectively extracts an optical characteristic with respect to at least one image of said plurality of images; and
the single imaging means detects the plurality of separately formed images, respectively and separately.

7. An optical image measuring device comprising:
single imaging means;
a combination of optical elements forming a plurality of light paths which separate a single image to be detected onto a single imaging means, forming images thereof in the same imaging direction; and
a wavelength selecting element positioned in at least one of the light paths, for selecting a particular wavelength component;
wherein said single imaging means detects a plurality of images including different wavelength components obtained by separating the single image to be detected.

8. The optical image measuring device according to claim 6 or 7, wherein said imaging means comprises image processing means for performing image data processing by recognizing a plurality of images individually; and said image processing means performs positional alignment between respective images or image evaluation, on the basis of markers contained in the respective images.

9. A measurement method wherein light is irradiated onto an organism, the light emitted from the organism is detected by a two-dimensional detector, and biological information is measured using the detected images; comprising the steps of:
obtaining measurement image data for a plurality of pixels of an organism using a plurality of measurement wavelengths; and reference image data for at least one pixel using the same measurement wavelengths as the aforementioned measurement wavelengths; and
deriving two-dimensional information for absolute quantities relating to biological information by performing calculation based on the difference of said measurement image data and reference image data for each measurement wavelength.

10. The optical biological measurement method according to claim 9, wherein said plurality of measurement wavelengths are at least two wavelengths between 400 nm and 1000 nm.

11. The optical biological measurement method according to claim 9, wherein reference image data is obtained by detecting a reference subject using a plurality of measurement wavelengths and by means of a plurality of pixels, independently of measurement of the organism, and said calculation is performed with respect to the pixels of measurement image data of organism and the corresponding pixels of the reference image data.

12. The optical biological measurement method according to claim 9, wherein a single reference subject having time independent reflectivity is placed on a portion of the organism, the image data at a single location is detected using a plurality of measurement wavelengths, image data for a single pixel or image data derived from a plurality of pixels is taken as a single reference image data, and said calculation is performed with respect to each pixel of the measurement image data and the single reference image data.

13. The optical biological measurement method according to claim 9, wherein a plurality of reference subjects are placed on the organism, image data at a plurality of locations is detected using a plurality of measurement wavelengths, two-dimensional reference image data is determined by interpolating the image data at a plurality of locations, and said calculation is performed with respect to the pixels of the measurement image data and the corresponding pixels of the reference image data.

14. The optical biological measurement method according to claim 9, wherein image data detected using a plurality of measurement wavelengths at a location on the organism experiencing little time change is taken as a single reference image data, and said calculation is performed with respect to each pixel of the measurement image data and the single reference image data.

15. The optical biological measurement method according to claim 9 or 10, wherein the value relating to biological information is the quantity of oxy-haemoglobin or the quantity of the deoxy-haemoglobin.

16. The optical biological measurement method according to claim 9, wherein said calculation is performed taking the values relating to biological information and variation that is not dependent on wavelength as variables, to obtain values relating to the biological information from which variation not dependent upon wavelength has been removed.

17. An optical biological measurement device for irradiating light onto an organism, detecting the light emitted from the organism by a two-dimensional detector, and measuring biological information by means of the detected image; comprising:
image measuring means for measuring two-dimensional measurement image data for the organism using a plurality of measurement wavelengths; and
image calculating means, having reference image data for at least one pixel obtained using the same measurement wavelength as said measurement wavelength, for determining two-dimensional information for absolute quantities relating to biological information, by performing calculation based on the difference of said measurement image data and reference image data for each measurement wavelength.

18. The optical image measuring device according to claim 5, wherein said inducing means for inducing a change in the state of the organism is one of the following, a cuff for halting blood flow, a heater in order to increase the skin temperature, ice or cooling means in order to reduce the skin temperature, a device for applying electrical stimulus, a lamp to radiate the skin or medicines for inducing a change in blood flow.
